(19) United States
(12) Patent Application Publication
(10) Pub. No.: US 2010/0021480 A1
(43) Pub. Date: Jan. 28, 2010

(54) BIOACTIVE SUBSTANCE-BLOOD PROTEIN CONJUGATE AND STABILIZATION OF A BIOACTIVE SUBSTANCE USING THE SAME

(75) Inventors: Jong-Il Park, Daejeon-City (KR); Min-Gyu Soung, Daejeon-City (KR); Heung-Jae Kim, Daejeon-City (KR); Chae-Jin Lim, Daejeon-City (KR); Jong-Phil Kang, Daejeon-City (KR); Ho-Il Choi, Daejeon-City (KR)

Correspondence Address: LEXYOUME IP GROUP, LLC, 5180 PARKSTONE DRIVE, SUITE 175, CHANTILLY, VA, 20151, US

(73) Assignee: PEPTRON CO., LTD, DAEJEON-CITY (KR)

(21) Appl. No.: 12/090,969

(22) PCT Filed: Oct. 27, 2006
(86) PCT No.: PCT/KR2006/004427

§ 371 Date: Apr. 21, 2008

Related U.S. Application Data
(60) Provisional application No. 60/731,592, filed on Oct. 27, 2005.

(51) Int. Cl. A61K 39/395; C07K 14/435; C07K 14/76; C07K 16/00; A61K 38/16; A61P 35/00

(52) U.S. Cl. 424/178.1; 530/380; 530/362; 530/391.1; 514/12

(57) ABSTRACT

This invention relates to a technology of modifying low-molecular-weight bioactive substances with short in vivo half-life and low stability in order to achieve a stable and efficient in vivo delivery thereof. More specifically, the present invention relates to a stable bioactive substance-blood protein conjugate, wherein a low-molecular-weight bioactive substance is ex vivo conjugated with a specific functional group on a blood protein through a reactive group, the low-molecular-weight bioactive substance is available as a drug for treatment and prevention in mammals including human and selected from the group consisting of a natural substance; and a method of a stable and efficient in vivo delivery of the low-molecular-weight bioactive substance based on the use of the bioactive substance-blood protein conjugate.

### CROSS REFERENCE TO RELATED APPLICATION

This application claims priority to and the benefit of Provisional Patent Application No. 60/731,592 filed on Oct. 27, 2005, which is hereby incorporated by reference for all purposes as if fully set forth herein.

### BACKGROUND OF THE INVENTION

(a) Field of the Invention

This invention relates to a technology of modifying low-molecular-weight bioactive substances with short in vivo half-life and low stability in order to achieve a stable and efficient in vivo delivery thereof. More specifically, the present invention relates to a stable bioactive substance-blood protein conjugate, wherein a low-molecular-weight bioactive substance is ex vivo conjugated with a specific functional group on a blood protein through a reactive group, the low-molecular-weight bioactive substance is available as a drug for treatment and prevention in mammals including human and selected from the group consisting of a natural substance; and a method of a stable and efficient in vivo delivery of the low-molecular-weight bioactive substance based on the use of the bioactive substance-blood protein conjugate.

(b) Description of the Related Art

With the introduction of insulin, biopharmaceutics opened and has rapidly developed along with the advancement of life science and completion of human genome project and since 2000, biopharmaceutics more than 500 has been on clinical studies and approximately 10 treatments are approved by Food and Drug Administration (FDA) every year. In particular, among the biopharmaceutics, peptide-based medicines are characterized by their strong treatment and preventive effects and biocompatibility, thereby being studied as new treatments or alternative treatments in the field of treatment and prevention for numerous disease symptoms.

However, as such peptide drugs and unstable low-molecular-weight drugs are easily biodegraded by various enzymes such as proteases present in vivo, they usually have short half-life. Furthermore, in the case of the peptide-class drugs, it is especially difficult to maintain their efficient concentration in blood in comparison with other low molecular drugs. Also, because they are macromolecules, their penetration into biological membranes is not easy, they may cause immunogenicity, and they generally have low solubility and thus their formulation has numerous restrictions. In particular, of the above mentioned drawbacks, the short half-life, low in vivo stability, and low bioavailability (BA) are recognized as portions to be improved in the development of prevention and treatment agents.

In general, most medicines provide their medicinal components into body in oral or injection form and they can exert treatment and prevention effects only when they are present within blood above a certain concentration. In many cases, high dose is given in order to increase the effect of treatment but it frequently involves various side effects and thus its use has the limits.

There have been proposed administration methods based on various drug delivery systems (DDS) such as slow-release capsule, depot and pump to improve the above-mentioned problems. However, such approaches have too many drawbacks to be generally applied so as to maintain the concentration of treatment medicines for a long-time period. For example, in case of skin adhesion dosage forms, they have to possess properties capable of penetrating medicines into skin tissues at a suitable rate when adhered to skin, and in slow-release dosage forms, dosage particles have time constraint for their release and are rapidly eliminated by macrophages when run into blood. If treatment medicines are to be administered by injection, many cases where prescription for injection is required repeatedly and continuously would not be advisable. In particular, self administration causes side effects due to its unskilled handling and in many cases, exhaustive personal training about administration methods is needed.

The development of drug delivery system which can efficiently improve the drawbacks about the drugs themselves, i.e., short half-life, low in vivo stability and low bioavailability (BA) and repetitive injection administration method over long time period is desperately required.

### SUMMARY OF THE INVENTION

It is an object of the present invention to provide a method of stabilizing a low-molecular-weight bioactive substance which is useful in the body, by ex vivo conjugating the low-molecular-weight bioactive substance to a blood protein using a reactive group capable of forming a stable bond between the low-molecular-weight bioactive substance and a specific functional group on the blood protein, to improve the stability and pharmacokinetic properties of a low-molecular-weight bioactive substance.

It is another object of the invention to provide a low-molecular-weight bioactive substance-blood protein conjugate, wherein a low-molecular-weight bioactive substance which is useful in the body and a blood protein are conjugated through a stable covalent bond.

It is still another object of the invention to provide a composition and a method for in vivo delivery of a low-molecular-weight bioactive substance, using the low-molecular-weight bioactive substance-blood protein conjugate, thereby increasing the in vivo half-life of the low-molecular-weight bioactive substance and improving the stability thereof.

It is still another object of the present invention to provide a composition and a method for treating a mammal disease by administering the low-molecular-weight bioactive substance-blood protein conjugate with improved in vivo stability.

It is still another object of the invention to provide a method for simple and efficient in vitro analysis comprising the step of conducting in vitro analysis of a stable bond between the specific functional group of the blood protein and the low-molecular-weight bioactive substance in quantitative and qualitative ways.

### BRIEF DESCRIPTION OF THE DRAWINGS

FIG. 1 is a graph showing long-acting degree of blood glucose reducing effects for 10 hours, comparing Compound 35 of the invention with native Exendin-4 through intraperitoneal glucose tolerance test (IPGTT).

control: administered with phosphate buffer saline;

each sample: subcutaneously administered once with Exendin-4 and Compound 35, respectively, at 10 hours before the test start;

glucose is intraperitoneally administered to all groups at 0 min.; and

the number of mice in each group: 4.

FIG. 2 is a graph showing long-acting degree of blood glucose reducing effects for 24 hours, comparing Compound 35 of the invention with Exendin-4 through intraperitoneal glucose tolerance test (IPGTT).

control: administered with phosphate buffer saline;

each sample: subcutaneously administered once with Exendin-4, and Compound 35, respectively, at 24 hours before the test start;

glucose is intraperitoneally administered to all groups at 0 min.; and

the number of mice in each group: 4.

### DETAILED DESCRIPTION OF THE PREFERRED EMBODIMENTS

A more complete appreciation of the invention, and many of the attendant advantages thereof, will be readily apparent as the same becomes better understood by reference to the following detailed description.

This invention relates to a technology of conjugating a low-molecular-weight bioactive substance which is useful in the body to a blood protein using a reactive group capable of forming a stable bond between the low-molecular-weight bioactive substance and a specific functional group on the blood protein, thereby increasing the stability of the low-molecular-weight bioactive substance.

In one aspect, the present invention provides a method of stabilizing a low-molecular-weight bioactive substance, comprising the steps of:

1) linking a proper functional group to the low-molecular-weight bioactive substance with or without a linking group, to prepare a modified bioactive substance;

2) linking a proper reactive group to a functional group for binding on a blood protein, to activate the functional group; and

3) ex vivo reacting the modified bioactive substance and the blood protein with the activated functional group, thereby forming a stable bond between the bioactive substance and the blood protein, to generate a stable bioactive substance-blood protein conjugate.

By the method of the present invention, it can be achieved that the low-molecular-weight bioactive substance which is unstable in vivo aqueous circumstance, especially in blood, can be stabilized, and its in vivo half-life and staying time can be extended, to effectively exhibit the inherent functions thereof.

More specifically, the method of stabilizing a low-molecular-weight bioactive substance of the present invention may comprise the steps of:

reacting a functional group on a blood protein, which is selected from the group consisting of a hydroxyl group (—OH), a thiol group (—SH), an amino group (—NH₂), and a carboxyl group (—CO₂H), with a reactive group which is capable of forming a stable covalent bond with the functional group, to activate the blood protein; and

ex vivo reacting the activated blood protein with a low-molecular-weight bioactive substance having molecular weights of 100,000 or less which may be selected from the group consisting of a natural peptides, synthetic peptides, natural hormones, synthetic hormones, and raw materials for drugs, to form a stable covalent bond therebetween. In the method of the present invention, the reactive group may be released, after the stable covalent bond between the blood protein and the low-molecular-weight bioactive substance is formed.

In another aspect, the present invention provides a low-molecular-weight bioactive substance-blood protein conjugate formed by a stable covalent bond between the low-molecular-weight bioactive substance and a functional group on the blood protein, wherein the functional group is selected from the group consisting of a hydroxyl group (—OH), a thiol group (—SH), an amino group (—NH₂), and a carboxyl group (—CO₂H), and the blood protein is activated by a reactive group which is capable of forming a stable covalent bond with the functional group, whereby the stability of the bioactive substance is improved.

In another aspect, the present invention provides a method of in vivo delivery of bioactive substances, and a method of treating or preventing diseases against which the bioactive substances have therapeutic activities, using the low-molecular-weight bioactive substance-blood protein conjugate. In addition, the present invention provides a composition for in vivo delivery of bioactive substances, and a composition for treating or preventing diseases against which the bioactive substances have therapeutic activities, containing the low-molecular-weight bioactive substance-blood protein conjugate.

In the present invention, the low-molecular-weight bioactive substance includes all the natural or synthetic organic compounds and natural or synthetic peptides, exhibiting the effects to improve, treat, or prevent against the symptoms and diseases of mammals, especially human, and in particular, having a molecular weight of 100,000 or less. More specifically, the low-molecular-weight bioactive substance of the present invention may be one or more selected from the group consisting of natural substances, natural peptides, synthetic peptides, natural hormones, synthetic hormones, and raw materials for drugs. For example, the low-molecular-weight bioactive substance of the present invention may include insulinotropic peptides, such as, glucagons like peptide-1(GLP-1), glucagon family peptide hormones, such as, exendin-3 or exendin-4, which have blood glucose reducing effects, or Luteinizing Hormone-Releasing Hormone (LHRH).

In an embodiment of the present invention, the low-molecular-weight bioactive substance may be ex vivo conjugated with albumin by reactive groups capable of forming a stable covalent disulfide (S—S) bond with free thiol groups (Cys³⁴) of albumin, preferably albumin prepared by gene recombination techniques, to improve the pharmacokinetic properties (half-life, in vivo stability, etc.) of the low-molecular-weight bioactive substance. In a preferred embodiment of the present invention, in order to effectively form a stable covalent bond, especially a stable covalent disulfide (S—S) bond, between the low-molecular-weight bioactive substance and the blood protein outside the body (ex vivo), the blood protein may be activated by the reactive groups. For example, a free thiol group on cystein located at the 34^{th} position of albumin which is one of the blood proteins may be a disulfanyl group which is one of the reactive groups, to effectively induce a bond between the low-molecular-weight bioactive substance and the blood and thereby to more effectively stabilize the bioactive substance.

The reactive group may include a binding part capable of forming a stable covalent bond with a specific functional group on the blood protein and a leaving group to be released therefrom after forming the stable covalent bond. In an embodiment of the present invention, the blood protein may be albumin, preferably albumin prepared by gene recombination techniques, and the stable covalent bond may be a disulfide (S—S) bond with free thiol groups (Cys³⁴) on the albumin. In another embodiment of the present invention, a linking group may be additionally used to link the bioactive substance and the reactive group.

Preferably, the blood protein may be albumin, preferably albumin prepared by gene recombination techniques. For example, albumin activated by binding of a disulfanyl group as a reactive group to a free thiol group on cystein which is the 34^{th} amino acid residue of albumin may be designed and used.

In another aspect, the present invention provides a stabilized bioactive substance-blood protein conjugate, wherein a low-molecular-weight bioactive substance useful in the body and a blood protein are conjugated through a stable covalent bond. In an embodiment of the present invention, in the bioactive substance-blood protein conjugate, a stable disulfide (S—S) covalent bond is formed between the bioactive substance and a free thiol group on cystein which is the 34^{th} amino acid residue of albumin. In another embodiment of the present invention, the albumin may be activated so as to effectively form a ‘stable disulfide covalent bond’ with the low-molecular-weight bioactive substance. For example, the albumin may be activated by a reactive group. Preferably, a free thiol group on cystein which is the 34^{th} amino acid residue of albumin may be activated by a disulfanyl group which is one of the reactive groups. In still another embodiment of the present invention, the bioactive substance-blood protein conjugate may additionally comprise a proper linker group.

In another aspect, the present invention provides a method of preparing the bioactive substance-blood protein conjugate. In a preferable embodiment, the method may include the steps of: 1) linking a proper functional group to a low-molecular-weight bioactive substance with or without a linking group, to prepare a modified bioactive substance; 2) linking a proper reactive group to a blood protein, to activate a binding functional group on the blood protein; and 3) ex vivo reacting the modified bioactive substance and the activated blood protein, to form a stable covalent bond between the bioactive substance and the blood protein. Preferably, the blood protein may be albumin, especially, albumin prepared by gene recombination techniques, and for example, albumin activated by binding of a disulfanyl group as a reactive group to a free thiol group on cystein which is the 34^{th} amino acid residue of albumin may be designed and used. As mentioned above, the albumin is activated by the activated free thiol group, thereby having an increased ex vivo binding ability to the low-molecular-weight bioactive substance, to be able to form a stable S—S covalent bond with the low-molecular-weight bioactive substance.

In still another aspect, the present invention provides a composition for delivery of the bioactive substance with extended half-life and improved stability into blood (in vivo), containing the bioactive substance-blood protein conjugate, and a method of in vivo delivery of the bioactive substance with improved stability using the bioactive substance-blood protein conjugate.

The method of the present invention is characterized in that the low-molecular-weight bioactive substance with low stability and short half-life is stabilized, not by administering the bioactive substance into blood, and then, allowing it to bind to a blood protein in vivo, but by conjugating the bioactive substance to the blood protein ex vivo through a stable covalent bond to form a stable conjugate. When in vivo stabilizing the low-molecular-weight bioactive substance by administering the bioactive substance into blood and allowing it to bind to a blood protein in vivo, there are several problems such that the binding degree of the bioactive substance and the blood protein is low, resulting in a low stability of the bioactive substance, and the free bioactive substance which is not binding to the blood protein may invading the brain, causing various brain-associated diseases, such as Alzheimer's disease. However, in the present invention, the bioactive substance-protein blood conjugate is formed and stabilized ex vivo, and all the bioactive substance molecules are conjugated with the blood protein, resulting in advantages that the in vivo stability of the bioactive substance is considerably increased when administered into blood, any free bioactive substance is generated, and there is no possibility to cause brain-associated diseases.

In still another aspect, the present invention provides a pharmaceutical composition containing the bioactive substance-protein blood conjugate as an active ingredient, and a diagnosing or treating method by administering the bioactive substance-protein blood conjugate in an efficient amount to a patient in need of administration of the bioactive substance. The diagnosing or treating activity of the present method depends on the inherent activity of the bioactive substance. For example, in the case of the use of a insulinotropic peptide, such as glucagons like peptide-1 (GLP-1) and the like, or a glucagon family peptide hormone such as exendin-3, exendin-4, and the like, as the bioactive substance, the diagnosing or treating activity may be the activity for the blood glucose reducing or against the diseases caused by the increase of the blood glucose, such as diabete mellitus. Further, in the case of the use of luteinizing hormone-releasing hormone (LHRH), the diagnosing or treating activity may be the activity against the diseases associated with sex hormone-related diseases and control the ovulation period in mammals, for example, the activity to diagnose of incompetence of the functions of hypothalamus, hypophysis, and reproductive organs, and to treat the diseases, such as prostate cancer, and endometriosis.

In still another aspect, the present invention provides a modified albumin wherein the free thiol group on cystein, the 34^{th} amino acid of albumin, is activated by binding of a reactive group selected from the group consisting of 2-pyridyl disulfanyl group, N-alkylpyridinium disulfanyl group, 5-nitro-2-pyridyl disulfanyl group, 3-nitro-thiophenyl disulfanyl, 1-piperido disulfanyl group, 3-cyano-propyl disulfanyl group, 2-thiouredyl disulfanyl group, 4-carboxylbenzyl disulfanyl group, 1-phenyl-1H-tetrazolyl disulfanyl group, 1-amino-2-naphthyl disulfanyl group, 3-carboxyl-6-pyridyl disulfanyl group, 2-benzothiazolyl disulfanyl group, and 4-nitro-thiophenyl disulfanyl group.

It is still another aspect, the invention provides a method for simple and efficient in vitro analysis comprising the step of conducting in vitro analysis of a stable bond between the specific functional group on blood protein and a low-molecular-weight bioactive substance in quantitative and qualitative ways.

### 1. Definition of Terms

1) Bioactive Substances: As used herein, the term “bioactive substances” refers to all nature-derived or synthetic organic compounds and nature-derived or synthetic peptides having improvement, treatment and prevention effects on symptoms or diseases in mammals, especially, human, and in particular, to low molecular substances having molecular weights of 100,000 or less. More particularly, the bioactive substances of the invention may be nature-derived natural substances, peptides, hormones, synthetic peptides, synthetic hormones and raw medicinal materials. For example, there may be included insulinotropic peptides such as glucagons like peptide-1 (GLP-1), glucagon family peptide hormones such as exendin-3 or exendin-4, which have blood glucose reducing effects in mammals, and LHRH (Luteinizing Hormone-Releasing Hormone). The administration of the bioactive substances together with the bioactive substance carriers or the bioactive substance-bioactive substance carrier conjugates according to the invention enables the bioactive substances to exert their inherent in vivo activity in more efficient way.

Glucagon like peptide-1 (GLP-1): GLP-1 is a intestinal hormone peptide that consists of 31 amino acids, and is released from proglucagon produced in L-cells of GI-tract. It decreases the blood glucose level by stimulating insulin depending on the concentration of glucose in blood, delays empty feeling in stomach, decreases intake of foods, and in particular, stimulates the functions of β-cells. Accordingly, the administration of the bioactive substance-bioactive substance carrier conjugates wherein GLP-1 is bound as a bioactive substance or GLP-1 together with the bioactive substance carriers can result in excellent blood glucose reducing effects, whereby high blood glucose-related diseases such as diabetes or obesity can be effectively treated or prevented.

Exendin-3 and Exendin-4 peptide: Exendin-3, Exendin-4 and derivatives thereof are poison components of Heloderma suspectum and they are nature-derived peptides consisting of 39 amino acids with blood glucose reducing effects. Accordingly, the administration of the bioactive substance-bioactive substance carrier conjugates wherein Exendin-3, Exendin-4 or derivatives thereof is bound as a bioactive substance or Exendin-3, Exendin-4 or derivatives thereof together with the bioactive substance carriers can result in excellent blood glucose reducing effects, whereby high blood glucose-related diseases such as diabetes can thus be effectively treated or prevented.

LHRH (Luteinizing Hormone-Releasing Hormone): LHRH is a hormone generated in hypothalamus stimulates the release of follicle-stimulating hormone (FSH) and luteinizing hormone (LH) from the anterior lobe of the pituitary. The agents of acetate thereof are effectively used to diagnose whether hypothalamus, pituitary gland and genital organs have abnormal or inactive functions and recently, they are used as treatments for disease symptoms such as prostate cancer, endometriosis and uterus myoma. Accordingly, the administration of the bioactive substance-bioactive substance carrier conjugates wherein LHRH is bound as a bioactive substance or LHRH together with the bioactive substance carriers can play roles to treat or diagnose sex hormone-related diseases and control the ovulation period in mammals, and further, the diseases such as prostate cancer, endometriosis and uterus myoma can be effectively treated or prevented thereby.

Modified bioactive substance: As used herein, the term “modified bioactive substances” refers to all compounds designed by binding a proper functional group capable of ex vivo conjugating with a reactive group on an activated blood protein, preferably a substituted-disulfanyl group. Generally, such modified bioactive substances are designed so as to be stable against various peptidases, which is resulted from the fact that modified bioactive substances can be present in a conjugate (complex) form by conjugation with the substituted-disulfanyl group on the activated blood (plasma) protein through ‘new and stable disulfide covalent bonds.’ In addition, in the present invention, the bioactive substance may comprise free thiol groups so that such stable disulfide covalent bonds can be quantitatively measured.

The modified bioactive substances to be mainly used in the present invention may include natural substances, synthetic organic compounds, nature-derived peptides, synthetic peptides and the like, having molecular weights of 100,000 or less with pharmacological activity that can be used with certain treatment or prevention purpose in mammals, preferably human. The modified bioactive substances may usually be linked to the functional groups through linker groups, or directly linked to the functional group without linker groups. Also, in the present invention, the bioactive substance may be modified such that the ex vivo formation of selective ‘S—S covalent bond’ between the bioactive substance and the activated albumin can be directly or indirectly analyzed by simple in vitro qualitative and quantitative methods.

The modified bioactive substance may be in the form of the bioactive substance-functional group or the bioactive substance-linker group-functional group complex. 2) Reactive group: As used herein, the term “reactive group” means all the chemical groups capable of forming a new and stable covalent bond with specific functional groups on blood proteins, for example, hydroxyl group (—OH), thiol group (—SH), amino group (—NH₂) or carboxyl group (—CO₂H), preferably “S—S covalent bond” with free thiol group (—SH group) on plasma proteins. In preferred embodiments, the representative chemical reactive group present on the activated serum albumin may be a substituted-disulfanyl group, and it may react with a free thiol group, which is the functional group present on the modified bioactive substance, in aqueous solution or ex vivo environment, to form a new and stable disulfide covalent bond. The reactive group of the present invention may be selected from the group consisting of disulfanyl groups, and the disulfanyl groups may include 2-pyridyl disulfanyl group, N-alkylpyridinium disulfanyl group, 5-nitro-2-pyridyl disulfanyl group, 3-nitro-thiophenyl disulfanyl, 1-piperido disulfanyl group, 3-cyano-propyl disulfanyl group, 2-thiouredyl disulfanyl group, 4-carboxylbenzyl disulfanyl group, 1-phenyl-1H-tetrazolyl disulfanyl group, 1-amino-2-naphthyl disulfanyl group, 3-carboxyl-6-pyridyl disulfanyl group, 2-benzothiazolyl disulfanyl group, 4-nitro-thiophenyl disulfanyl group, and the like. The reactive group may optionally contain a leaving group that is separated after the formation of the stable S—S bond by reaction with the free thiol groups.

3) Linker group: As used herein, the term “linker group” refers to all the chemical moieties that can be linked or bound to both of the free thiol group on the bioactive substance. The linker group may include alkyl groups of C1 to C6 consisting of one or more methyl, ethyl, propyl, butyl, etc., alkoxy group, cycloalkyl group, polycyclic group, aryl group, polyaryl group, substituted aryl group, heterocyclic group and substituted heterocyclic group. In addition, the linker group may include poly ethoxy amino acids including 2-amino (ethoxy) acetic acid (AEA). The preferable linker group of the invention may be AE(E)ₙA ([2-(2-amino)-ethoxy](ethoxy)ₙ acetic acid) (n=0˜2) containing one to three ethoxy groups. In particular, the use of AEEE acetic acid (AEEEA) increases of solubility, resulting in advantageous effects on the formation of stable covalent bonds between the bioactive substances and blood components, thereby exhibiting better blood glucose reducing effects than AEEA.

The linker group can link the bioactive substance and the reactive group by being bound to the terminal of the substance or positioning the inside of the substance.

4) Functionality: In the present invention, the term “functionality” can be defined as functional groups on the modified bioactive substance, which can form a new and stable covalent bond, especially ‘S—S covalent bond’ by reacting with the reactive group on the blood protein, especially the activated albumin. In general, various functional groups, such as, hydroxyl group (-OH), thiol group (—SH), amino group (—NH₂), carboxyl group (—CO₂H), and the like, may be present on the modified bioactive substance. In a preferable embodiment of the present invention, free thiol group (—SH), which may be located at the C-terminus, N-terminus or inside of the modified bioactive substance, and the reactive group on the activated albumin may react, to form a new and stable disulfide covalent bond.

5) Blood components: In the present invention, the blood components may be present in the mobile or fixed form in blood. The fixed blood components may include tissues membrane receptors, interstitial proteins, fibrin proteins, collagens, platelets, endothelial cells and epithelial cells that have no mobility in blood. Further, they may also include cell membranes, membrane receptors, somatic body cells, skeletal, smooth muscle cells, neuronal components, osteocytes and osteoclasts that are associated with the above examples. The mobile blood components are blood components capable of continuously locomoting without being fixed. In general, they are not associated with cell membranes and are present in the concentration of at least 0.1 ug/ml in blood. The blood protein components, which can be used as mobile blood components in the invention, may include serum albumin, transferrin, ferritin, celuroplasmin and immunoglobulin such as IgM and IgG. Generally, in vivo half-life of the mobile blood components is at least 12 hours. In addition, the blood components may include albumin prepared by gene recombination techniques.

6) Plasma protein: The plasma proteins mean all the proteins that are contained in plasma. Most plasma proteins present in blood are serum albumin and globulin. Approximately 7 g is contained within 100 uM of plasma. Albumin is contained in 50 to 70%, globulin is in approximately 20 to 50%, and fibrinogen is within 10%. The blood protein that does not contain the fibrinogen is called as ‘serum protein’.

The plasma protein to be mainly used in the present invention may include transferrin, IgG, celuroplasmin, serum albumin, and the like, and among them, serum albumin is preferable. When the human being is selected as a subject to be administered, human serum albumin (HAS), preferably HAS extracted from human blood or prepared by gene recombination techniques, may be used. When other mammals than human is selected as a subject, serum albumin of the selected mammal, preferably serum albumin extracted from the mammal blood or prepared by gene recombination techniques, may be used.

9) Protective group: In the present invention, the term “protective group” can be defined as a chemical functional group derived from the reaction among amino acids in the synthesis of peptides and its representative examples may include acetyl (Ac), fluorenylmethyloxy-acrbonyl (Fmoc), t-butyloxycarbonyl (Boc), benzyloxycarbonyl (Cbz), t-butyl (t-Bu), tri-phenylmethyl (Trt), 2,2,4,6,7-pentamethyldihydrobenzofuran-S-sulfonyl (Pbf), and the like. The general protective groups and abbreviations of the amino acids used in the present invention are summarized in Table 1 below.

TABLE 1
Natural amino acids and their abbreviations
Natural amino acids and their abbreviations

| | 3-Letter | 1-Letter | |
|---|---|---|---|
| Name | abbreviation | abbreviation | Protected amino acids |
| Alanine | Ala | A | Fmoc-Ala-OH |
| Arginine | Arg | R | Fmoc-Arg(Pbf)-OH |
| Asparagine | Asn | N | Fmoc-Asn(Trt)-OH |
| Aspartic acid | Asp | D | Asp(tBu)-OH |
| Cysteine | Cys | C | Fmoc-Cys(Trt)-OH |
| Glutamic acid | Glu | E | Fmoc-Glu(tBu)-OH |
| Glutamine | Gln | Q | Fmoc-Gln(Trt)-OH |
| Glycine | Gly | G | Fmoc-Gly-OH |
| Histidine | His | H | Fmoc-His(Trt)-OH |
| Isoleucine | Ile | I | Fmoc-Ile-OH |
| Leucine | Leu | L | Fmoc-Leu-OH |
| Lysine | Lys | K | Fmoc-Lys(Boc)-OH |
| Methionine | Met | M | Fmoc-Met-OH |
| Phenylalanine | Phe | F | Fmoc-Phe-OH |
| Proline | Pro | P | Fmoc-Pro-OH |
| Serine | Ser | S | Fmoc-Ser(tBu)-OH |
| Threonine | Thr | T | Fmoc-Thr(tBu)-OH |
| Trytophan | Trp | W | Fmoc-Trp(Boc)-OH |
| Tyrosine | Tyr | Y | Fmoc-Tyr(tBu)-OH |
| Valine | Val | V | Fmoc-Val-OH |

### 2. Structural Form and Construction of Modified Bioactive Substances and Activated Albumin

The structural form and constitution of the modified bioactive substances and the activated albumin of the present invention can be illustrated as follows:

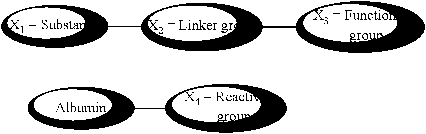

### <Structural Form and Construction of Modified Substance and Activated Albumin>

The “X₁, bioactive substances” are reaction compounds with molecular weights of 100,000 or less which exhibit physiological activity, and may refer to nature-derived natural substances, peptides, hormones, synthetic peptides, synthetic hormones and raw medicinal substances, which have pharmacological effects and can be efficiently used in the treatment and/or prevention against diseases in mammals, preferably human. For example, the bioactive substances may include insulinotropic peptides such as glucagons like peptide-1 (GLP-1), glucagon family peptide hormones, such as exendin-3 or exendin-4, luteinizing hormone-releasing hormone (LHRH), and the like.

The “X₂, linker group” refers to a linker group of positioning between the bioactive substances and the reactive group and connecting them through a chemical bond. The linker group may include alkyl group of C1 to C6 consisting of one or more methyl, ethyl, propyl, butyl, etc., alkoxy group, cycloalkyl group, polycyclic group, aryl group, polyaryl group, substituted aryl group, heterocyclic group, substituted heterocyclic group, and the like. In addition, the linker group may include poly ethoxy amino acids, such as (2-amino) ethoxy acetic acid (AEA). The preferred linker group of the invention may be AE(E)ₙA ([2-(2-amino)-ethoxy](ethoxy)n acetic acid) (n=0˜2) containing one to three ethoxy groups. In particular, the use of AEEE acetic acid (AEEEA) increases solubility and has advantageous effects on the formation of stable covalent bonds between the bioactive substances and blood components, and thus, better blood glucose reducing effects can be obtained than AEEA.

“X₃, functional group” refers to a functional group on the modified bioactive substance capable of forming a new and stable disulfide covalent bond by reacting a reactive group on activated albumin. Generally, various groups, such as hydroxyl group(—OH), thiol group(—SH), amino group(—NH₂) and carboxyl group(—CO₂H), are present on the modified bioactive substance. However, in the present invention, free thiol group (—SH), which may be located at the C-terminus, N-terminus or inside of the modified bioactive substance, and the reactive group on the activated albumin may react, to form a new and stable disulfide covalent bond.

The “X₄, reactive group” refers to all the chemical groups which are bound on the activated serum albumin, and capable of forming a new and stable covalent bond with specific functional groups on modified bioactive substance, such as, hydroxyl group (—OH), thiol group (—SH), amino group (—NH₂) or carboxyl group (—CO₂H), preferably “S—S covalent bond” with free thiol group (—SH group) on the modified bioactive substance. In preferred embodiments, the reactive group on the activated serum albumin may be a substituted-disulfanyl group, which can react with a free thiol group (—SH group) on the modified bioactive substance in aqueous solutions or ex vivo environment, to form a new and stable disulfide covalent bond. For example, the reactive groups may include 2-pyridyl disulfanyl group, N-alkylpyridinium disulfanyl group, 5-nitro-2-pyridyl disulfanyl group, 3-nitro-thiophenyl disulfanyl, 1-piperido disulfanyl group, 3-cyano-propyl disulfanyl group, 2-thiouredyl disulfanyl group, 4-carboxylbenzyl disulfanyl group, 1-phenyl-1H-tetrazolyl disulfanyl group, 1-amino-2-naphthyl disulfanyl group, 3-carboxyl-6-pyridyl disulfanyl group, 2-benzothiazolyl disulfanyl group, 4-nitro-thiophenyl disulfanyl group, and the like. The chemical structure of the above-mentioned reactive groups may be shown in the following formula 1, and they may optionally comprise a leaving group that is separated after the reaction with a free thiol group.

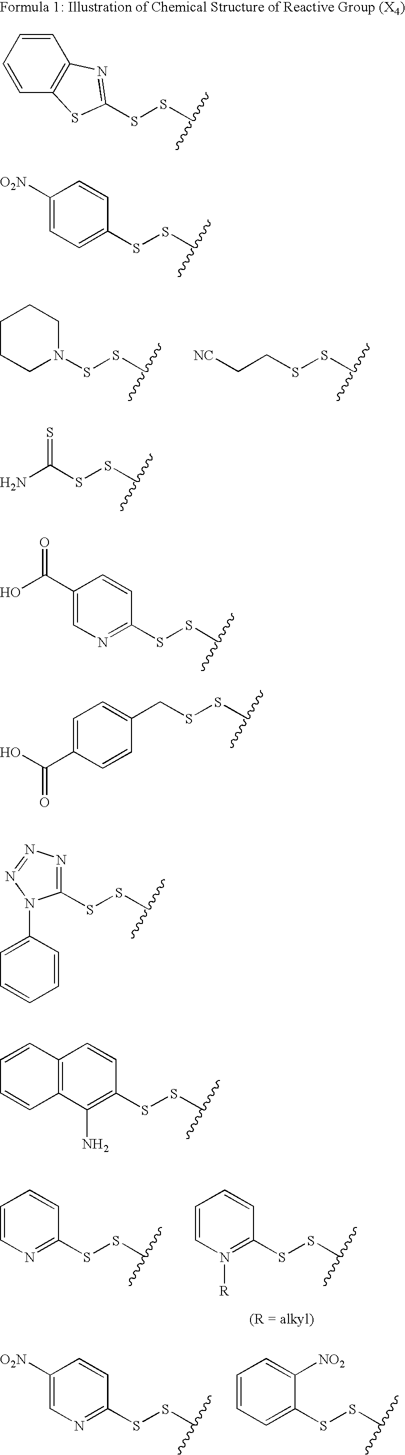

### 3. Synthesis of Active Albumin

The plasma protein to be mainly used in the present invention may include transferrin, IgC, celuroplasmin, serum albumin, and the like, and among them, serum albumin is preferable. When the human being is selected as a subject to be administered, human serum albumin (HAS), preferably HAS extracted from human blood or prepared by gene recombination techniques, may be used. When other mammals than human is selected as a subject, serum albumin of the selected mammal, preferably serum albumin extracted from the mammal blood or prepared by gene recombination techniques, may be used.

In a preferable embodiment, the present invention provides a effective method of modifying functional groups on albumin so that free thiol group located at the 34^{th} amino acid of human serum albumin (HSA), cystein (Cys³⁴), can be selectively conjugated with the modified bioactive substance with free thiol groups, in aqueous solution or buffer solution environment.

### 4. Albumin Binding Test

It can be shown by measuring the binding degree of albumin, that the modified bioactive substance of the present invention can be ex vivo conjugated with the substituted disulfanyl group on the activated albumin, whereby the in vivo stability of the bioactive substance increases. In addition, the comparison of the albumin-binding degree of the modified bioactive substance of the present invention to that of the natural bioactive substance with no modification, can show the fact that the modified bioactive substance of the present invention can be more effectively conjugated with albumin, whereby the stability thereof increases, compared with the natural bioactive substance. Such binding degree to albumin can be simply qualitatively or quantitatively measured by an in vitro analysis using a simple HPLC.

The conventional analysis used to determine whether albumin and bioactive substances are conjugated to create a conjugation complex had experimental limits and problems where albumin complex needs to be separately purified and analyzed using LC-MS and MALDI-TOF. However, the albumin binding test provided by the present invention is experimentally meaningful in that the conjugation can be in vitro measured through a simple pre-treatment and HPLC analysis.

The result of the measurement of the conjugation degree with increasing the concentration of the bioactive substance at the fixed concentration of albumin suggests the fact that the degree of the disulfide conjugation is deemed to be closely related to the condition of albumin, especially, to the content thereof with respect to free thiol groups.

### 5. Quantitative Analysis of Albumin-Bioactive Substance Conjugate

The present invention may also provide a method for effective in vitro quantitative analysis with regard to the disulfide complex wherein albumin and modified bioactive substance are ex vivo conjugated with each other through disulfide bond.

The conventional analysis used to determine whether albumin and bioactive substances are conjugated to create a conjugation complex had experimental limits and problems where albumin complex needs to be separately purified and analyzed using LC-MS and MALDI-TOF. However, in the present invention, the disulfide bond between the conjugation complex may be selectively reduced by treatment of DTT (dithiothreitol; Cleland's reagent), thereby easily measuring the amount of the bioactive substances that are separated and released from the complex. This analysis has significant meaning in experiment in that the bioactive substances in conjugation with albumin can be effectively measured in vitro through HPLC analysis.

In the present invention, a quantitative analysis with treatment of DTT may be performed to determine the disulfide conjugation between the albumin and the bioactive 5 substance.

The present invention is further explained in more detail with reference to the following examples. These examples, however, should not be interpreted as limiting the scope of the present invention in any manner.

### Example

### Example 1

### Compound 1: D-Ala⁸-GLP-1 (7-36)-Lys³⁷-[ε-AEEA-CO(CH₂)₂—SH]—NH₂. 4TFA His-D-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Lys-[ε-AEEA-CO(CH₂)₂—SH]—NH₂.4TFA (SEQ ID NO: 1)

100 μmol of rink amide methylbenzhydrylamine (MBHA) resin (0.6 mmol/g, Novabiochem Corporation) were measured and put into a reaction vessel. The resin was solvated with 5 ml of DMF and allowed to be sufficiently swollen for 5 min. 3 ml of 20% piperidine DMF solution was added to the swollen resin, which was then shaken for 10 minutes, and the piperidine solution was removed therefrom. Then, 20% piperidine DMF solution was added again and reaction was kept for 10 min, thereby completely eliminating Fmoc protective group that protected the resin, and then, washed five times or more with 10 ml of DMF solvent. In this step, it was determined using Kaiser Test whether the Fmoc protective group is deprotected [E. Kaiser et al., Anal. Biochem. (1970) 34, 595].

Fmoc-Lys(Aloc)-OH (500 μmol), HOBt (500 μmol), HBTU (500 μmol) and DIEA (1 mmol) were completely dissolved in 5 ml of DMF solvent, and then, added to the resin which is deprotected from Fmoc protective group. The reaction solution was shaken at room temperature for 2 hours or so, and then, washed with 10 ml of DMF solvent five times or more. In this step, Kaiser Test was performed in the same way as above to determine whether the coupling of Fmoc-amino acids occurs.

Next, coupling was successively carried out in accordance with the following synthesis cycle: (1) washing with DMF solvent (10 ml) five times or more; (2) deprotecting using 20% piperidine DMF solution (3 ml) two times for 10 min; (3) washing with DMF solvent (10 ml) five times or more; (4) adding Fmoc-amino acid; (5) activating the amino acid and coupling for 2 hours by addition of coupling reagent; and (6) washing with DMF solvent (10 ml) five times or more.

Step 1: Coupling Step

Amino acids (5 equivalents or more) protected with Fmoc were added to the resin reaction vessel in the following order and coupled: (1) Fmoc-Lys(Aloc)-OH; (2) Fmoc-Arg(Pbf)-OH; (3) Fmoc-Gly-OH; (4) Fmoc-Lys(tBoc)-OH; (5) Fmoc-Val-OH; (6) FmocLeu-OH; (6) Fmoc-Trp-OH; (7) Fmoc-Ala-OH; (8) Fmoc-Ile-OH; (9) Fmoc-Phe-OH; (10) Fmoc-Glu(OtBu)-OH; (11) Fmoc-Lys(tBoc)-OH; (12)Fmoc-Ala-OH; (13) Fmoc-Ala-OH; (14) Fmoc-Gln(Trt)-OH; (15) Fmoc-Gly-OH; (16) Fmoc-Glu(OtBu)-OH; (17) Fmoc-Leu-OH; (18) Fmoc-Tyr(tBu)-OH; (19) Fmoc-Ser(tBu)-OH; (20) Fmoc-Ser(tBu)-OH; (21) Fmoc-Val-OH; (22) Fmoc-Asp(OtBu)-OH; (23) Fmoc-Ser(tBu)-OH; (24) Fmoc-Thr(tBu)-OH; (25)-Fmoc-Phe-OH; (26) Fmoc-Thr(tBu)-OH; (27) Fmoc-Gly-OH; (28) Fmoc-Glu(OtBu)-OH; (29) Fmoc-D-Ala-OH; (30) Boc-His(N-Trt)-OH.

Step 2: Selective Deprotection Step of Aloc Group

After 300 μmol of Pd(PPh₃)₄ was dissolved in 5 ml of CH₃Cl:NMM:AcOH (18:1:0.5), it was added to the resin synthesized in Step 1 and shaken at room temperature for 2 hours or more. The reaction resin was washed with CHCl₃ (10 ml, six times); 20% acetic acid CH₂Cl₂ solution (10 ml, six times); CH₂Cl₂ (10 ml, six times); and DMF (10 ml, six times or more). The occurrence of the selective deprotection of Aloc group was determined by carrying out Kaiser Test in the same way as above.

Step 3: Introduction Step of Linker Group

After Fmoc-(AEEA)-OH (Fmoc-miniPEG-OH, 3 mmol), HOBt (3 mmol), HBTU (3 mmol) and DIEA (6 mmol) were completely dissolved in 10 ml of DMF solvent, and added to the resin which is deprotected from Fmoc protective group. The reaction solution was shaken at room temperature for 4 hours or more, and then, washed with 10 ml of DMF solvent ten times or more. In this step, Kaiser Test was performed in the same way as above to determine the occurrence of the coupling of Fmoc-amino acids. The reaction solution was treated with 10 ml of 20% piperidine DMF solution, shaken for 30 min or more thereby to remove Fmoc protective group, and then, washed with 10 ml of DMF five times or more.

After deprotection of Fmoc protecting group, 3-(Tritylthio)propionic acid(2 mmol), HBTU(2 mmol), HOBt(2 mmol) and DIEA(4 mmol) were completely dissolved in 10 ml of DMF solvent, and then, added to the synthesized resin. The reaction solution was shaken at room temperature for 4 hours or more, and then, washed with 10 ml of DMF solvent ten times or more.

Step 4: Cleavage Step

Immediately upon the completion of synthesis, the resin coupled with the peptides was cleaved by using the mixture of TFA/water (95:5) for 3 hours. The obtained mixture solution was treated with excessive amount of diethyl ether solvent that had been refrigerated, to generate a precipitate. The obtained precipitate was centrifuged so as to be completely precipitated, and the excessive amount of TFA was primarily eliminated, and these procedures were repeated two times or so, whereby solid peptides were obtained.

The obtained peptides were purified with HPLC using C-18 columns and an acetonitrile/water concentration gradient solvent system containing 0.01% TFA over 50 min, wherein the concentration gradient ranges from 5% to 100%. The purified pure fractions were lyophilized, to obtain Compound 1, D-Ala⁸-GLP-1 (7-36)-Lys³⁷[□-AEEA-CO—(CH₂)₂—SH]—NH₂.4TFA in the form of white powder of TFA salts:

### Compound 1: D-Ala⁸-GLP-1 (7-36)-Lys³⁷[ε-AEEA-CO(CH₂)₂—SH]—NH₂.4TFA MALDI-TOF=3,660

The synthesis of Compound 1 described in the above can be summarized as reaction formula 1 shown in below.

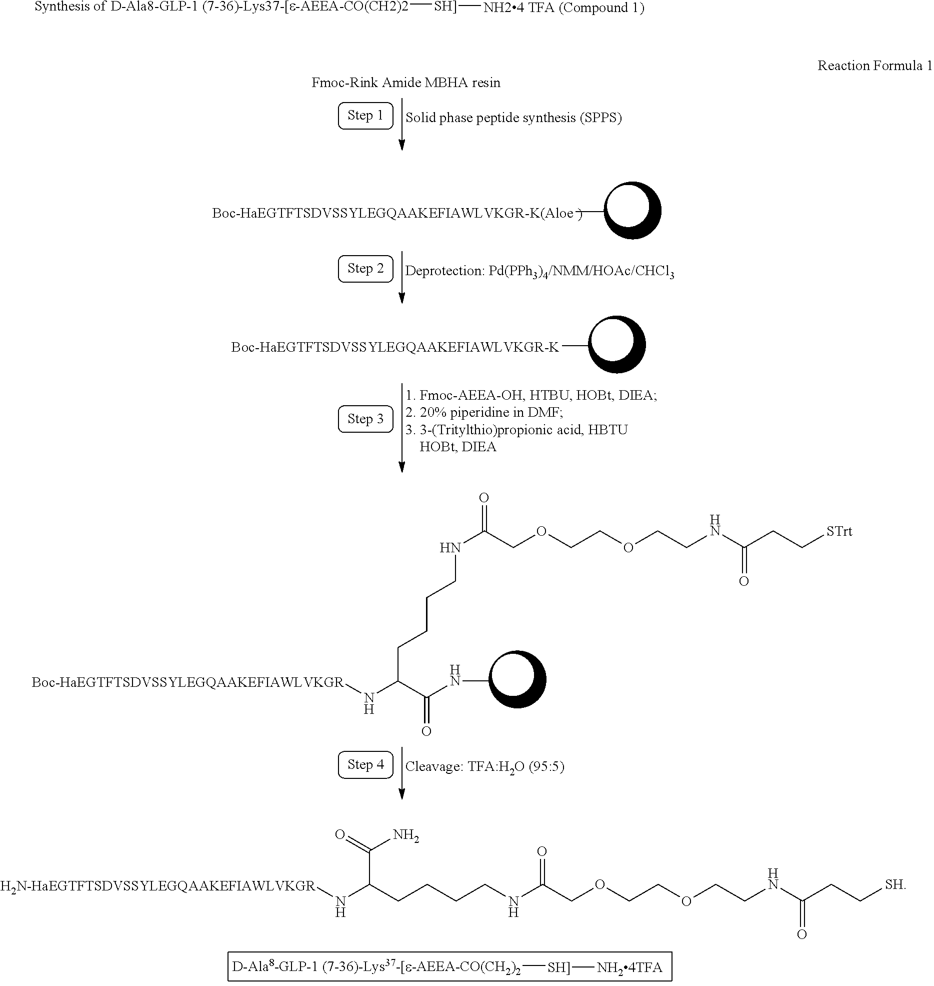

The following peptides were prepared in accordance with the same synthesis procedures as above.

### Compound 2: D-Ala⁸-GLP-1 (7-36)-Lys³⁷-[ε-AEEEA-CO—(CH₂)₂—SH]—NH₂.4TFA: His-D-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Lys-[ε-AEEEA-CO—(CH₂)₂—SH]—NH₂.4TFA (SEQ ID NO: 2)

Rt=23.93 min. (various concentration gradients ranging from 5% to 100% of acetonitrile/water containing 0.01% TFA over 30 min); MALDI-TOF=3,705.

### Compound 3: D-Ala⁸-Lys²⁶-[ε-AEEA-CO—(CH₂)₂—SH]-GLP-1 (7-36)-NH₂. 4TFA: His-D-Ala-Glu-Gly-Thr-Phe-Thr-Ser-Asp-Val-Ser-Ser-Tyr-Leu-Glu-Gly-Gln-Ala-Ala-Lys-[ε-AEEA-CO—(CH₂)₂—SH]-Glu-Phe-Ile-Ala-Trp-Leu-Val-Lys-Gly-Arg-NH₂.4TFA (SEQ ID NO: 3)

Rt=24.25 min. (various concentration gradients ranging from 5% to 100% of acetonitrile/water containing 0.01% TFA over 30 min); MALDI-TOF=3,532.

### Compound 4: D-Ala⁸-Lys²⁶-[ε-AEEEA-CO—(CH₂)₂—SH]-GLP-1 (7-36)-NH₂. 4TFA: His-D-Ala-Glu-Gly-Thr-Phe-Thr-Ser-Asp-Val-Ser-Ser-Tyr-Leu-Glu-Gly-Gln-Ala-Ala-Lys-[ε-AEEEA-CO—(CH₂)₂—SH]-Glu-Phe-Ile-Ala-Trp-Leu-Val-Lys-Gly-Arg-NH₂. 4TFA (SEQ ID NO: 4)

Rt=24.11 min. (various concentration gradients ranging from 5% to 100% of acetonitrile/water containing 0.01% TFA over 30 min); MALDI-TOF=3,577.

### Compound 5: GLP-1 (7-36)-Lys³⁷-[ε-AEEA-CO—(CH₂)₂—SH]—NH₂.4TFA: His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Lys-[ε-AEEA-CO—(CH₂)₂—SH]—NH₂.4TFA (SEQ ID NO: 5)

Rt=24.01 min. (various concentration gradients ranging from 5% to 100% of acetonitrile/water containing 0.01% TFA over 30 min); MALDI-TOF=3,660.

### Compound 6: GLP-1 (7-36)-Lys³⁷-[ε-AEEEA-CO—(CH₂)₂—SH]—NH₂.4TFA: His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Lys-[ε-AEEEA-CO—(CH₂)₂—SH]—NH₂.4TFA (SEQ ID NO: 6)

Rt=23.91 min. (various concentration gradients ranging from 5% to 100% of acetonitrile/water containing 0.01% TFA over 30 min); MALDI-TOF=3,705.

### Compound 7: Exendin-4 (1-39)-Lys⁴⁰-[68 -AEEA-CO—(CH₂)₂—SH]—NH₂.5TFA: His-Gly-Glu-Gly-Thr-Phe-Thr-Ser-Asp-Leu-Ser-Lys-Gln-Met-Glu-Glu-Glu-Ala-Val- Arg-Leu-Phe-Ile-Glu-Trp-Leu-Lys-Asn-Gly-Gly-Pro-Ser-Ser-Gly-Ala-Pro-Pro-Pro-Ser-Lys-[ε-AEEA-CO—(CH₂)₂—SH]—NH₂.5TFA (SEQ ID NO: 7)

Rt=16.95 min. (various concentration gradients ranging from 5% to 100% of acetonitrile/water containing 0.01% TFA over 30 min); MALDI-TOF =4,548.

### Compound 8: Exendin-4 (1-39)-Lys⁴⁰-[ε-AEEEA-CO—(CH₂)₂—SH]—NH₂.5TFA: His-Gly-Glu-Gly-Thr-Phe-Thr-Ser-Asp-Leu-Ser-Lys-Gln-Met-Glu-Glu-Glu-Ala-Val- Arg-Leu-Phe-Ile-Glu-Trp-Leu-Lys-Asn-Gly-Gly-Pro-Ser-Ser-Gly-Ala-Pro-Pro-Pro-Ser-Lys-[ε-AEEEA-CO—(CH₂)₂—SH]—NH₂.5TFA (SEQ ID NO: 8)

Rt=16.86 min. (various concentration gradients ranging from 5% to 100% of acetonitrile/water containing 0.01% TFA over 30 min); MALDI-TOF=4,592.

### Compound 9: Lys²⁷-[ε-AEEA-CO—(CH₂)₂—SH]-Exendin-4 (1-39)-NH₂.5TFA: His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-[ε-AEEA-CO—(CH₂)₂—SH]-Asn-Gly-Gly-Pro-Ser-Ser-Gly-Ala-Pro-Pro-Pro-Ser-NH₂.5TFA (SEQ ID NO: 9)

Rt=21.41 min. (various concentration gradients ranging from 5% to 100% of acetonitrile/water containing 0.01% TFA over 30 min); MALDI-TOF =4,420.

### Compound 10: Lys²⁷-[ε-AEEEA-CO—(CH₂)₂—SH]-Exendin-4 (1-39)-NH₂.5TFA: His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-[ε-AEEEA-CO—(CH₂)₂—SH]-Asn-Gly-Gly-Pro-Ser-Ser-Gly-Ala-Pro-Pro-Pro-Ser-NH₂.5TFA (SEQ ID NO: 10)

Rt=21.48 min. (various concentration gradients ranging from 5% to 100% of acetonitrile/water containing 0.01% TFA over 30 min); MALDI-TOF =4,465.

### Compound 11: Exendin-3 (1-39)-Lys⁴⁰-[ε-AEEA-CO—(CH₂)₂—SH]—NH₂.5TFA: His-Ser-Asp-Gly-Thr-Phe-Thr-Ser-Asp-Leu-Ser-Lys-Gln-Met-Glu-Glu-Glu-Ala-Val- Arg-Leu-Phe-Ile-Glu-Trp-Leu-Lys-Asn-Gly-Gly-Pro-Ser-Ser-Gly-Ala-Pro-Pro-Pro-Ser-Lys-[ε-AEEA-CO—(CH₂)₂—SH]—NH₂.5TFA (SEQ ID NO: 11)

Rt=21.21 min. (various concentration gradients ranging from 5% to 100% of acetonitrile/water containing 0.01% TFA over 30 min); MALDI-TOF =4,564.

### Compound 12: Exendin-3 (1-39)-Lys⁴⁰-[ε-AEEEA-CO—(CH₂)₂—SH]—NH₂.5TFA: His-Ser-Asp-Gly-Thr-Phe-Thr-Ser-Asp-Leu-Ser-Lys-Gln-Met-Glu-Glu-Glu-Ala-Val- Arg-Leu-Phe-Ile-Glu-Trp-Leu-Lys-Asn-Gly-Gly-Pro-Ser-Ser-Gly-Ala-Pro-Pro-Pro-Ser-Lys-[ε-AEEEA-CO—(CH₂)₂—SH]—NH₂.5TFA (SEQ ID NO: 12)

Rt=21.18 min. (various concentration gradients ranging from 5% to 100% of acetonitrile/water containing 0.01% TFA over 30 min); MALDI-TOF=4,6094.

### Compound 13: Lys²⁷-[ε-AEEA-CO—(CH₂)₂—SH]-Exendin-3 (1-39)-NH₂.5TFA: His-Ser-Asp-Gly-Thr-Phe-Thr-Ser-Asp-Leu-Ser-Lys-Gln-Met-Glu-Glu-Glu-Ala-Val- Arg-Leu-Phe-Ile-Glu-Trp-Leu-Lys-[ε-AEEA-CO—(CH₂)₂—SH]-Asn-Gly-Gly-Pro-Ser-Ser-Gly-Ala-Pro-Pro-Pro-Ser-NH₂.5TFA (SEQ ID NO: 13)

Rt=21.12 min. (various concentration gradients ranging from 5% to 100% of acetonitrile/water containing 0.01% TFA over 30 min); MALDI-TOF=4,436.

### Compound 14: Lys²⁷-[ε-AEEEA-CO—(CH₂)₂—SH]-Exendin-3 (1-39)-NH₂.5TFA: His-Ser-Asp-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-[ε-AEEEA-CO—(CH₂)₂—SH]-Asn-Gly-Gly-Pro-Ser-Ser-Gly-Ala-Pro-Pro-Pro-Ser-NH₂.5TFA (SEQ ID NO: 14)

Rt=21.10 min. (various concentration gradients ranging from 5% to 100% of acetonitrile/water containing 0.01% TFA over 30 min); MALDI-TOF=4,481.

### Example 2

### Compound 15: Leuprolide-GSG-Lys-[ε-AEEA-CO—(CH₂)₂—SH]—NH₂.2TFA: Pyr-His-Trp-Ser-Tyr-D-Leu-Leu-Arg-Pro-Gly-Ser-Gly-Lys-[ε-AEEA-CO—(CH₂)₂—SH]—NH₂.2TFA (SEQ ID NO: 15). (Pyr=pyroglutamic acid, pE)

100 μmol of rink amide MBHA resin (0.6 mmol/g, Novabiochem Corporation) were measured and put into a reaction vessel. The resin was solvated with 5 ml of DMF and allowed to be sufficiently swollen for 5 min. 3 ml of 20% piperidine DMF solution was added to the swollen resin, which was then shaken and the piperidine solution was removed therefrom. Then, 20% piperidine DMF solution was added again and reaction was kept for 10 min, thereby completely eliminating Fmoc protective group that protected the resin and then, washed five times or more with 10 ml of DMF solvent.

Fmoc-Lys(Aloc)-OH (500 μmol), HOBt (500 μmol), HBTU (500 μmol) and DIEA (500 μmol) were completely dissolved in 5 ml of DMF solvent, and then, added to the resin which is deprotected from Fmoc protective group. The reaction solution was shaken at room temperature for 2 hours or so and then, washed with 10 ml of DMF solvent five times or more. In this step, Kaiser Test was performed in the same way as above to determine whether the coupling of Fmoc-amino acids occurs.

Next, coupling was successively carried out in accordance with the following synthesis cycle: (1) washing with DMF solvent (10 ml) five times or more; (2) deprotecting using 20% piperidine DMF solution (3 ml) two times for 10 min; (3) washing with DMF solvent (10 ml) five times or more; (4) adding Fmoc-amino acid; (5) activating the amino acid and coupling for 2 hours by addition of coupling reagent; and (6) washing with DMF solvent (10 ml) five times or more.

Step 1: Coupling Step

Amino acids (5 equivalents or more) protected with Fmoc were added to the resin reaction vessel in the following order and coupled: (1) Fmoc-Lys(Aloc)-OH; (2) Fmoc-Gly-OH; (3) Fmoc-Ser(tBu)-OH; (4) Fmoc-Gly-OH; (5) Fmoc-Pro-OH; (6) Fmoc-Arg(Pbf)-OH; (6) Fmoc-Leu-OH; (7) Fmoc-D-Leu-OH; (8) Fmoc-Tyr(tBu)-OH; (9) Fmoc-Ser(tBu)-OH; (10) Fmoc-Trp(Boc)-OH; (11) Fmoc-His(Trt)-OH; (12) Boc-Pyr(tBu)-OH.

Step 2: Selective Deprotection Step of Aloc Group

After 300 μmol of Pd(PPh₃)₄ was dissolved in 5 ml of CH₃Cl:NMM:AcOH (18:1:0.5), it was added to the resin synthesized in Step 1 and shaken at room temperature for 2 hours or more. The reaction resin was washed with CHCl₃ (10 ml, six times); 20% acetic acid CH₂Cl₂ solution (10 ml, six times); CH₂Cl₂ (10 ml, six times); and DMF (10 ml, six times or more). The occurrence of the selective deprotection of Aloc group was determined by carrying out Kaiser Test in the same way as Example 1.

Step 3: Introduction Step of Linker Group

Fmoc-(AEEA)-OH (Fmoc-miniPEG-OH, 3 mmol), HOBt (3 mmol), HBTU (3 mmol) and DIEA (6 mmol) were completely dissolved in 10 ml of DMF solvent, and added to the resin deprotected from Fmoc protective group. The reaction solution was shaken at room temperature for 4 hours or more and then washed with 10 ml of DMF solvent ten times or more. In this step, Kaiser Test was performed in the same way as above to determine the occurrence of the coupling of Fmoc-amino acids. The reaction solution was treated with 10 ml of 20% piperidine DMF solution, shaken for 30 min or more thereby to remove Fmoc protective group, and then, washed with 10 ml of DMF five times or more.

N-succinimidyl-3-(2-pyridyldithio) propionate (SPDP, 2 mmol) purchased from Pierce Biotechnology was dissolved in 5 ml of CH₂Cl₂ solvent, reacted with the resin synthesized as above for 3 hours or more with shaking, and then, and washed with CH₂Cl₂ (10 ml) six times or more.

Step 4: Cleavage Step

Immediately upon the completion of synthesis, the resin with the peptides coupled was cleaved using the mixture of TFA/water (95:5) for 3 hours. The obtained mixture solution treated with excessive amount of diethyl ether solvent that had been refrigerated to produce a precipitate. The obtained precipitate was centrifuged so as to be completely precipitated, the excessive amount of TFA was primarily eliminated, and these procedures were repeated two times or so, whereby solid peptides were obtained.

The obtained peptides were purified with HPLC using C-18 columns and an acetonitrile/water concentration gradient solvent system containing 0.01% TFA over 50 min, wherein the concentration gradient ranges from 5% to 100%. The purified pure fractions were lyophilized, to obtain bioactive substance Compound 15, Leuprolide-GSG-Lys-(□-AEEA-PDSP)-NH₂.2TFA in the form of white powder of TFA salts.

### Compound 15: Leuprolide-GSG-Lys-(ε-AEEA-PDSP)-NH₂.2TFA

Rt=23.63 min (various concentration gradients ranging from 5% to 100% of acetonitrile/water containing 0.01% TFA over 30 min); MS(ESI)m/e, [M+H]⁺=1853.

The synthesis of Compound 15 described in the above can be summarized as reaction formula 2 shown in below.

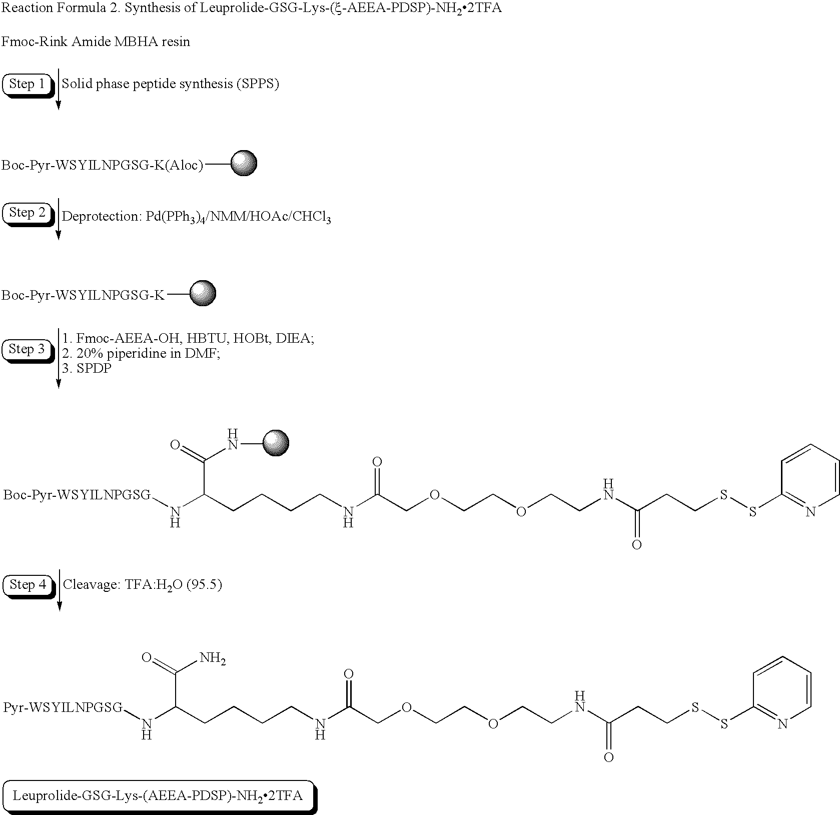

The following peptides were prepared in accordance with the same synthesis procedures as above.

### Compound 16: Leuprolide-GSG-Lys-(ε-AEEEA-PDSP)-NH₂.2TFA: Pyr-His-Trp-Ser-Tyr-Leu-Leu-Arg-Pro-Gly-Ser-Gly-Lys(ε-AEEEA-PDSP)-NH₂.2TFA (SEQ ID NO: 16). (Pyr=pyroglutamic acid)

### Compound 17: Leuprolide-GG-Lys-(ε-AEEA-PDSP)-NH₂.2TFA: Pyr-His-Trp-Ser-Tyr-D-Leu-Leu-Arg-Pro-Gly-Gly-Lys(ε-AEEA-PDSP)-NH₂.2TFA (SEQ ID NO: 17). (Pyr=pyroglutamic acid)

### Compound 18: Leuprolide-GG-Lys-(ε-AEEEA-PDSP)-NH₂.2TFA: Pyr-His-Trp-Ser-Tyr-D-Leu-Leu-Arg-Pro-Gly-Gly-Lys(ε-AEEEA-PDSP)-NH_{2.}2TFA (SEQ ID NO: 18). (Pyr=pyroglutamic acid)

### Compound 19: Leuprolide-Lys-(ε-AEEA-PDSP)-NH₂.2TFA: Pyr-His-Trp-Ser-Tyr-Leu-Leu-Arg-Pro-Lys(ε-AEEA-PDSP)-NH₂.2TFA (SEQ ID NO: 19). (Pyr=pyroglutamic acid)

### Compound 20: Leuprolide-Lys-(ε-AEEEA-PDSP)-NH₂.2TFA: Pyr-His-Trp-Ser-Tyr-Leu-Leu-Arg-Pro-Lys(ε-AEEEA-PDSP)-NH₂.2TFA (SEQ ID NO: 20). (Pyr=pyroglutamic acid)

### Example 3

### Preparation of Activated Albumin

HSA (500 mg, Sigma Aldrich) or albumin prepared by a gene recombination technique was dissolved in 10 ml of double distilled water, and then, the solution wherein aldrithiol (7.5 mg) was dissolved in 300 μl of CH₃CN was slowly added thereto at room temperature. The reaction solution was reacted with slowly shaking at room temperature for about 30 minutes. 10 μl of the reaction solution was collected and treated with Ellman's reagent (10 μl), to determine whether the free thiol group (Cys³⁴) of albumin is substituted by a new disulfanyl group through the change in the color of the Ellman's reagent.

The termination of the reaction can be determined by the change in the color of the Ellman's reagent, where the color of the Ellman's reagent is changed from achromatic color to dark yellow when the reaction is not completed, and the color of the Ellman's reagent is maintained as achromatic color when the reaction is completely terminated. After determining the termination of the reaction by the Ellman's test as above, the reaction product was lyophilized for at least 24 hours.

The lyophilized activated albumin was washed with MeOH(10 ml, three times), to remove the excess amount of aldrithiol and pyridyl-2-thione generated as a by-product. The obtained albumin sample was dissolved again in double distilled water, and then, lyophilized for at least 24 hours, to produce an activated albumin, which was called as ‘activated albumin 21.’

The examples of the activated albumins that can be produced by the above method are illustrated in the following Chemical Formula 2:

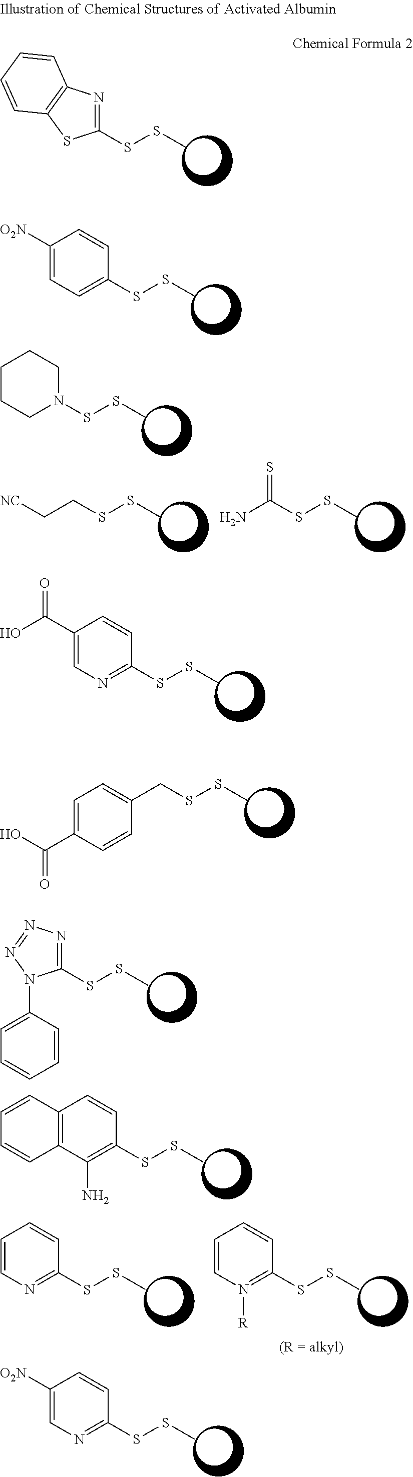

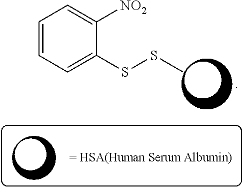

### Example 4

### Conjugation of Bioactive Substance Compound 15 (Leuprolide-GSG-Lys-[ε-AEEA-CO—(CH₂)₂—SH]—NH₂) and Activated Albumin

The present example provides an embodiment to effectively conjugate the modified bioactive substance compound 15 (Leuprolide-GSG-Lys-[□-AEEA-CO—(CH₂)₂—SH]—NH₂.2TFA) synthesized in Example 2 and the activated albumin 21 synthesized in Example 3 in PBS buffer solution.

### 4.1. Synthesis of Substrate-Albumin Conjugate 22:

The activated albumin 21(50 mg) synthesized in Example 3 was dissolved in PBS buffer solution (1 ml), and then, the solution wherein the modified bioactive substance compound 15 (2 mg) synthesized in Example 2 was dissolved in 100 μl of PBS buffer solution was slowly added thereto at room temperature. The reaction solution was reacted with slowly shaking at room temperature for about 30 minutes. 10 μl of the reaction solution was collected and treated with Ellman's reagent (10 μl), to determine whether the free thiol group (Cys³⁴) of albumin is substituted by a new disulfanyl group through the change in the color of the Ellman's reagent.

The termination of the reaction can be determined by the change in the color of the Ellman's reagent, where the color of the Ellman's reagent is changed from achromatic color to dark yellow when the reaction is not completed, and the color of the Ellman's reagent is maintained as achromatic color when the reaction is completely terminated. After determining the termination of the reaction by the Ellman's test as above, the reaction product was lyophilized for at least 24 hours.

The lyophilized activated albumin was washed with MeOH(10 ml, three times), to remove the unreacted compound 15 and pyridyl-2-thione generated as a by-product. The obtained albumin sample was dissolved again in double distilled water, and then, lyophilized for at least 24 hours, to produce an activated albumin in the crude form, which was called as ‘substrate-activated albumin 22.’

### 4.2. Purification of Substrate-Albumin Conjugate 22:

The substrate-albumin conjugate 22 synthesized by the above method may be purified using AKTA purifier(Amersham Biosciences, Uppsala, Sweden) under the following conditions: Firstly, a sodium phosphate buffer solution(20 mM, pH 7) consisting of sodium octanoate(5 mM) and (NH₄)₂SO₄(750 mM) was filled into 50 mL butyl sepharose 4 fast flow resin column (Amersham Biosciences, Uppsala, Sweden). Thereafter, substrate-albumin conjugate 22 was loaded thereto, and separated and purified at the flow rate of 2.5 ml per one minute.

Under such conditions, all desired substrate-albumin conjugates 22 are absorbed into hydrophobic resin, and the non-conjugated or unreacted HSA may be released and removed from the column. The purified substrate-albumin conjugate 22 was desalted, lyophilized for at least 24 hours, and then, stored in a freezer at -80° C. with being filled with nitrogen.

A variety of substrate-albumin conjugates can be created by the reaction, separation and purification methods as above. Substrate-albumin conjugates 22 to 41 which can be prepared using compounds 1 to 20 prepared in Examples 1 and 2 as above are illustrated in following Chemical Formula 3.

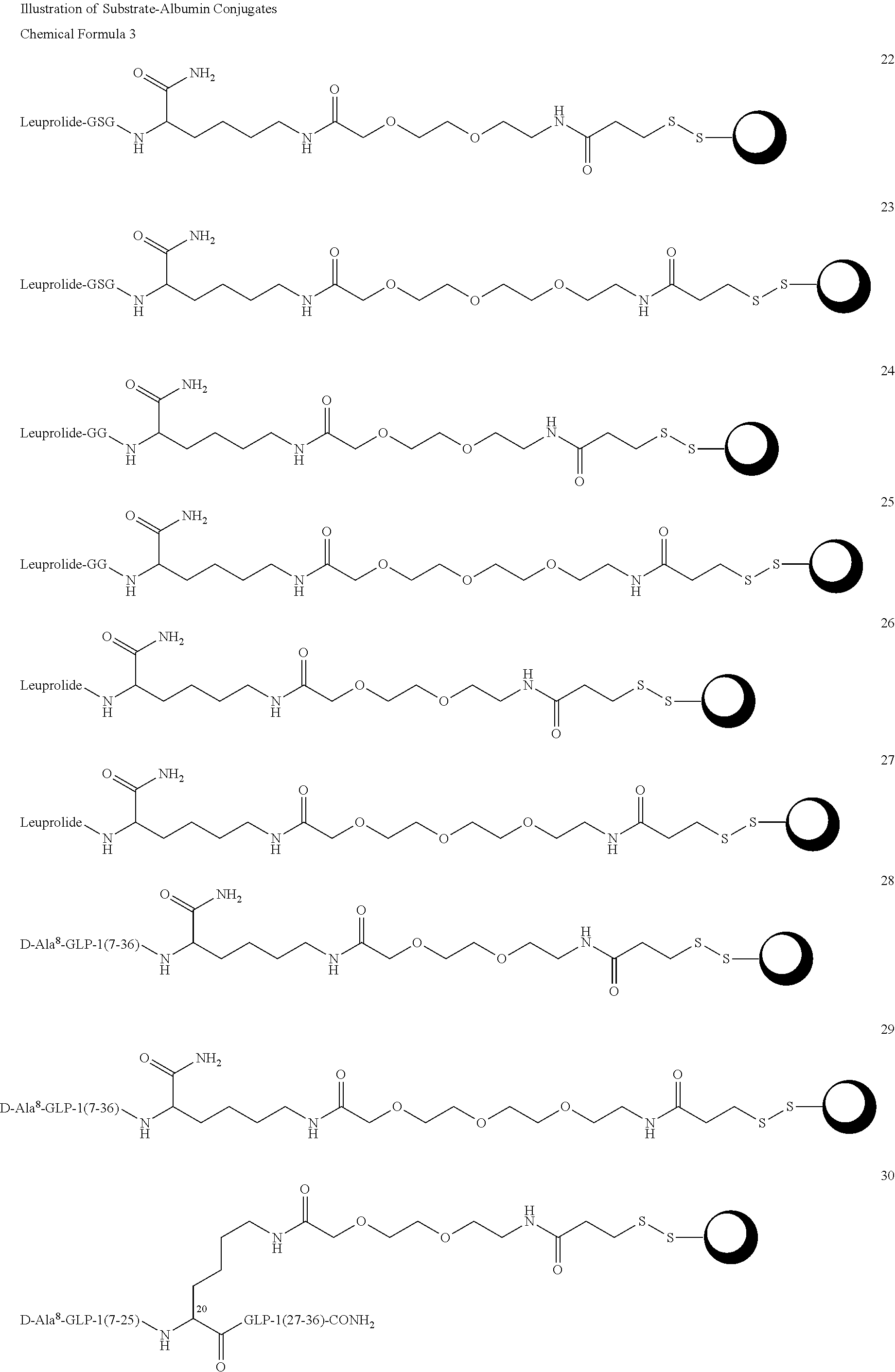

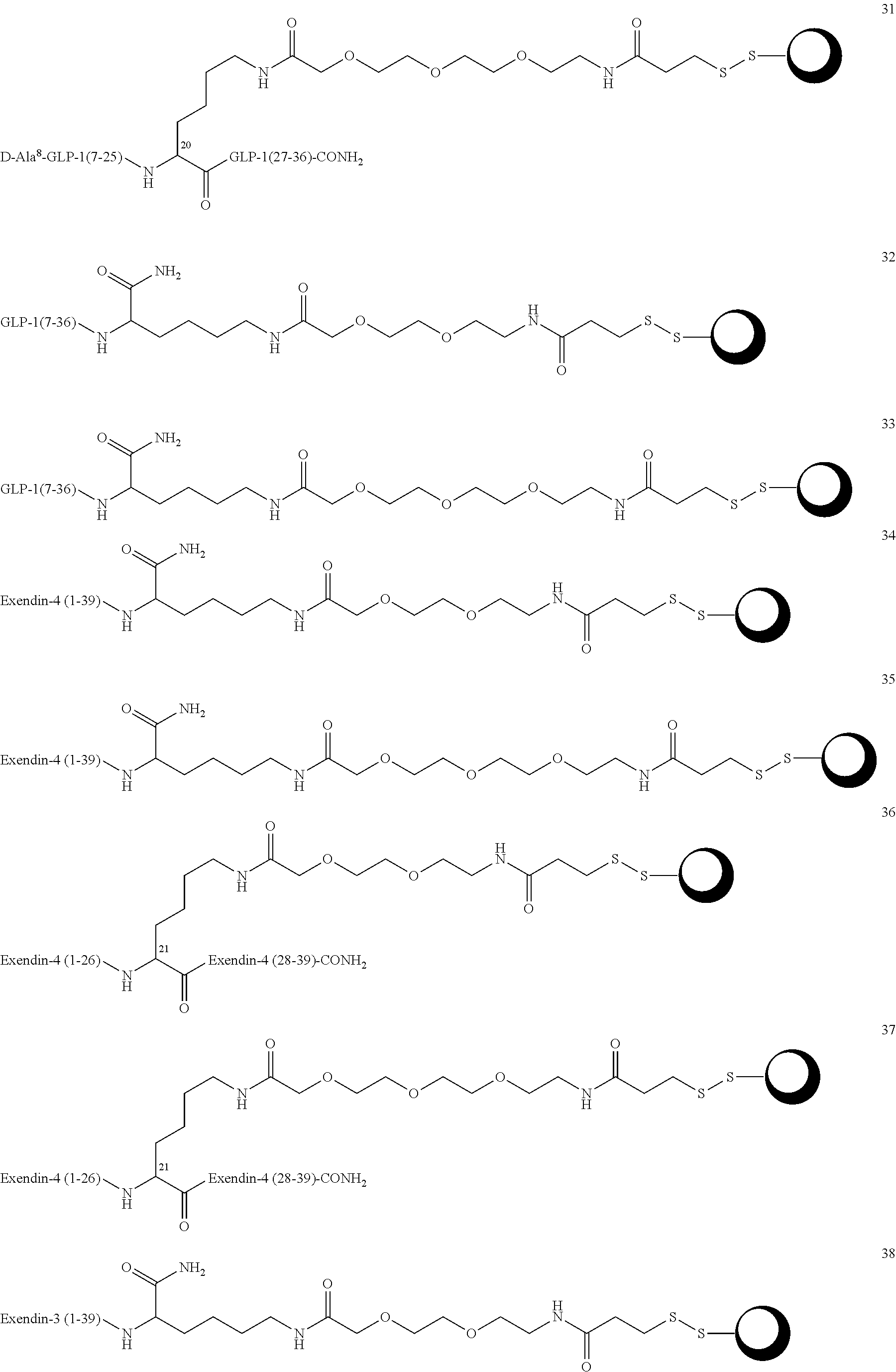

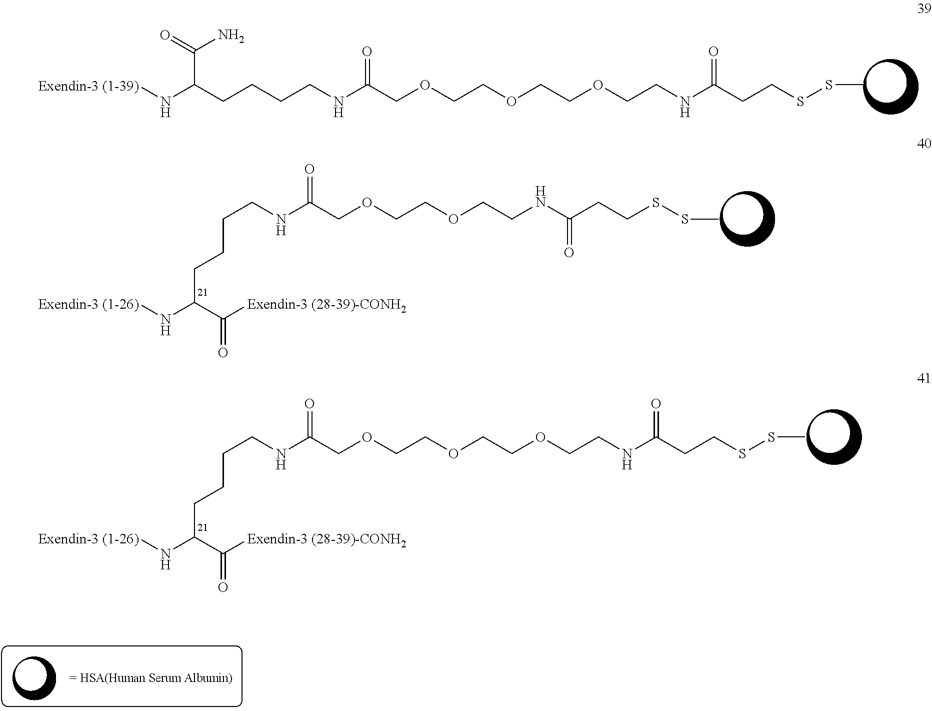

### Compound 35: MALDI-TOF=70,850

### Example 5

### Albumin Binding Test

It could be determined by measuring the degree of the bioactive substance-albumin conjugation that in vivo and ex vivo stabilities of Compound 15 (Leuprolide-GSG-Lys-[ε-AEEA-CO—(CH₂)₂—SH]—NH₂.2TFA), a modified bioactive substance synthesized in Example 2 above of the present invention, is increased by conjugation with a disulfanyl group on the activated HSA prepared in Example 3. Further, this experiment could show that the stability of Compound 15 synthesized according to the present invention has increased by more effectively binding to the activated albumin than unmodified Leuprolide.

### 5.1. Preparation of Stock Solutions:

Human serum albumin (HSA, 1 mM) solution was prepared by dissolving 66.5 mg of the activated HSA prepared in Example 3 in PBS buffer solution (pH 7.2, 1 ml). Stock solutions (1 mM) of bioactive substance Compound 15 (1.8 mg/ml) and Leuprolide (1.2 mg/ml) were respectively prepared by the same method.

### 5.2. Albumin Binding Test:

The stock solutions prepared in the above method were diluted with PBS buffer so that they had the compositions shown in Table 2 below thereby to prepare sample solutions (100 μl) for determination. Then, each reaction mixture was mixed and slowly shaken in an incubator for 30 min while the temperature condition of 37° C. was kept. After incubation, methanol (150 μl) was added to each sample vial, which was then voltexed for 10 min, to precipitate HSA. The precipitated albumin was spun down by centrifugation (12,000 rpm, 10° C., 10 min.) and the supernatants (50 μl) were taken and analyzed using HPLC under the same conditions.

TABLE 2
Composition of Stock Solutions

| | Activated Albumin | Compound 15 | Leuprolide | Buffer (PBS) |
|---|---|---|---|---|
| control | 50 μl | | 10 μl | 40 μl |
| 1 | 50 μl | 5 μl | — | 45 μl |
| 2 | 50 μl | 10 μl | — | 40 μl |
| 3 | 50 μl | 20 μl | — | 30 μl |
| 4 | 50 μl | 40 μl | — | 10 μl |
| 5 | 50 μl | 50 μl | — | — |
| 6 | 40 μl | 50 μl | — | 10 μl |
| 7 | 10 μl | — | — | 90 μl |
| 8 | — | 10 μl | | 90 μl |

### 5.3. Results:

The degree of conjugation was examined with increasing the concentration of the bioactive substance compounds at the fixed concentration of albumin. As the result, it could be considered that the degree of disulfide conjugation closely relates to the conditions of albumin used in the test, and in particular, it is associated with the content of the bioactive substance with respect to the free thiol groups. The results are summarized in Table 3 below.

TABLE 3
Albumin binding test Results

| Bioactive Substance | | PA^{20.6 min}/ | |
|---|---|---|---|
| Compound 15/albumin*¹ | PA^{20.6 min}*² | 4.4M*³ | |
| 5/50 | 0 | 0 | |
| 10/50 | 0 | 0 | |
| 20/50 | 1.3M | 0.3 | |
| 40/50 | 9.8M | 2.2 | |
| 50/50 | 14.7M | 3.3 | |
| 50/40 | 17.9M | 4.0 | |
| 10/0 | 4.4M | 1.0 | Compound 15 only |

*¹Albumin: Sigma A1653, remainder mostly globulins fraction V power albumin;
*²PA: peak area; and
*³M: million.

### Example 6

### Quantitative Analysis of Albumin-Bioactive Substance Conjugation Complex

The disulfide conjugate (complex) of the activated HSA and the modified bioactive substance Compound 15 (Leuprolide-GSG-Lys-[□-AEEA-CO—(CH₂)₂—SH]—NH₂.2TFA) which was prepared in Example 4 was treated with DTT (dithiothreitol; Cleland's reagent) to selectively reduced the bond present in the disulfide conjugate, whereby the amount of bioactive substance Compound 15 that is separated and released from the disulfide complex could be easily quantitatively measured. This analysis is experimentally meaningful in that bioactive substances which are conjugated with albumin can be effectively qualified in vitro through a simple method, such as HPLC analysis.

### 6.1. Preparation of Stock Solutions:

HSA (1 mM) solution was prepared by dissolving 66.5 mg of activated albumin 21 prepared in Example 3 in PBS buffer solution (pH 7.2, 1 ml). Stock solutions of 1 mM bioactive substance Compound 15 (1.8 mg/ml) and 100 mM DTT (15.4 mg/ml) were respectively prepared in accordance with the same method.

### 6.2. Quantitative Measurement of Albumin-Bioactive Substance Compound 15 Conjugation Complex:

Activated albumin solution (50 μl) and bioactive substance Compound 15 solution (10 μl) were mixed in each of 10 tubes, using the stock solutions prepared in the above method in the same conditions as used in the albumin binding test of Example 5, and then, incubated at 37° C. for 30 min while being slowly shaken.

After incubation, DTT was added thereto in amounts of 0 nmole, 100 nmole (2×), 200 nmole (4×), 500 nmole (10×), and 1,000 nmole, respectively, and reacted for about 1 hour at 37° C. After 1 hour elapsed, 25 μl was taken out from each sample, 50 μl of MeOH was added thereto, and the mixture was voltexed for 10 minutes, to precipitate HSA. The precipitated albumin was spun down by centrifugation (12,000 rpm, 10° C., 10 min), and the supernatants (50 μl) were taken and analyzed using HPLC under the same conditions.

Quantitative analysis procedures including treatment of DTT used to determine the presence of disulfide conjugation between albumin and the bioactive substances in the present invention can be illustrated as Reaction Formula 3 below.

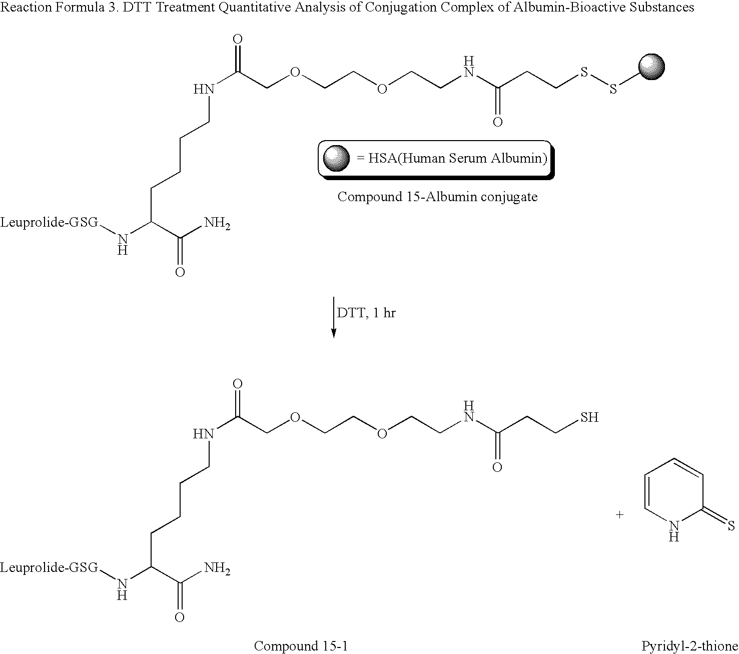

In the binding test of albumin and bioactive substance Compound 15, it was observed that Compound 15 was bound. In consideration of the characteristics of albumin and the test results, it is assumed that Compound 15 forms a disulfide conjugation by binding to Cys on the 34^{th} position of albumin. If albumin and Compound 15 are conjugated by disulfide bond as above-mentioned, it can be inferred that Compound 15-1 having free thiol group could be released from disulfide conjugation by treatment of DTT reagent of a suitable concentration. With this aim, albumin and Compound 15 were incubated for 30 min to form disulfide conjugation. Then, after the addition of four different amounts of DTT ranging from 100 nmole to 1,000 nmole, they were incubated for 1 hour.

### 6.3. Result

When treating with DTT at a high concentration of 1,000 nmole or higher, albumin protein was degraded by DTT, and thus, albumin precipitation was formed in large amounts, whereby the analysis and observation were not easy. The test results are shown in Table 4 below. As summarized in Table 4, Compound 15-1 that was expected to be released at all the DTT treatment concentrations was observed through HPLC analysis, and Compound 15-1 expected to be released was readily observed when treated with high concentration, in comparison with when treated with low concentration.

TABLE 4
Result of DTT Treatment

| Compound 15*¹/ | | |
|---|---|---|
| albumin*² | DTT (0.1 M) | Compound 15-1 |
| 10 μl/50 μl | 0 | Not detected |
| 10 μl/50 μl | 1 μl (2 X) | Detected |
| 10 μl/50 μl | 2 μl (4 X) | Detected |
| 10 μl/50 μl | 5 μl (10 X) | Detected |
| 10 μl/50 μl | 10 μl (20 X) | Detected |

*¹,*²1 mM in PBS;
*²Albumin: Sigma A1653, remainder mostly globulins fraction V power albumin.

### Example 7

### Animal Test: Test for Measuring Blood Glucose Reducing Activity through IPGTT

### 7.1. Methods

Long-acting of the blood glucose reducing effect was tested in this example to determine long-acting of the blood glucose reducing activity of Compound 35 of the present invention. As control samples for determining blood glucose reducing activity and long-acting of the activity, native Exendin-4 was used, and the activity of each peptide sample was measured by intraperitoneal glucose tolerance test (IPGTT).

ICR female mice (6 weeks old, DaehanBioLink, Korea) were employed as test animals, after being adapted in lab for 7 days. Before the test, 8 mice were picked out of each group and blood was collected from tail to measure the glucose concentration in blood using glucometer (Accucheck Sensor, Roche), and then, starved for 15 to 18 hours. Then, predetermined amount of each peptide sample was subtaneously administrated and after 4 hours or 9 hours, glucose (2 g/kg of mouse in PBS, pH 7.2) was intraperitoneally administrated (the time when glucose was administrated is defined as 0 min). At each determined time, blood was corrected from caudal vein and the level of glucose therein was measured with glucometer. The experiments for the determination of blood glucose reducing activity and the long-acting of the activity are summarized in Table 5.

TABLE 6
Blood glucose Decrease Activity Measurement Sample

| | | | Glucose |
|---|---|---|---|
| | | | Administration |
| Group¹ | Sample | Dose (nmol/kg) | Time (hr) |
| 1 | Control² | — | 10 hr, 24 hr |
| 2 | Exendin-4 | 10 | 10 hr, 24 hr |
| 3 | PDD5001 | 10 | 10 hr, 24 hr |

¹n = 8 in each group, and
²control: saline administering group

### 7.2. Results: Hypoglycemic Effects

This example is to determine the blood glucose activity of Compound 35 of the present invention, wherein the activity of each peptide sample was measured through intraperitoneal glucose tolerance test (IPGTT) measurement method using Native GLP-1, d-ala-GLP-1, Exendin-4 as control samples to determine long-acting of blood glucose decrease activity.

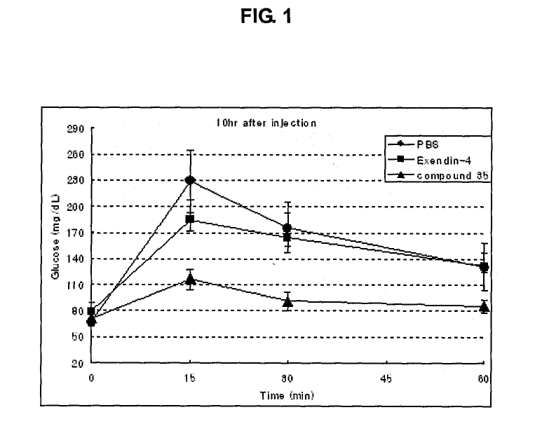

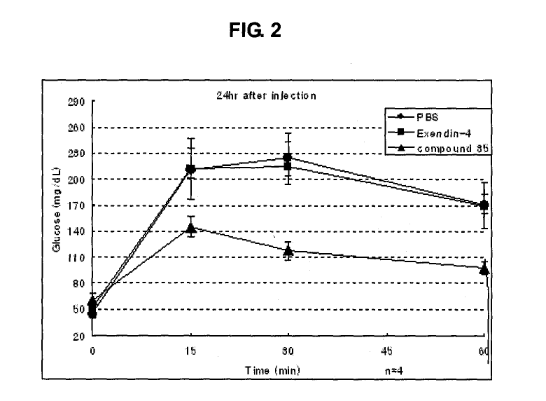

The animal test results with respect to the long-acting of blood glucose reducing effect, when Compound 35 of the present invention, and Exendin-4 were respectively administered into mice in the amount of 100 nmol/kg, were measured through IPGTT, and shown in FIGS. 1 and 2. In FIGS. 1 and 2, the control group was a group administrated with saline instead of Compound 35 or native Exendin-4, each group was subcutaneously administrated with each test compound before 10 or 24 hours, and then, was intraperitoneally administrated with glucose once at 0 min. The number of mice in each group was 8. The groups administrated with native Exendin-4 (10 nmol) showed no significant difference in blood glucose reducing curve from the control group administrated with saline only. In contrast, the group administrated with 10 nmol of Compound 35 showed significant reducing profile in blood glucose reducing curve after 24 hours as well as after 10 hours. Accordingly, Compound 35 has remarkably excellent blood glucose control effects in comparison with native Exendin-4 based on the dose of 10 nmol.

The result that Compound 35 has a superior blood glucose control effect in comparison with native Exendin-4 is considered to be caused by the fact that in vivo stability of Compound 35 is remarkably increased by in vivo conjugation of 2-pyridyl disulfanyl group of Compound 35 with free thiol group (Cys 34) of albumin via a new ‘disulfide covalent bond,’ when the compound was administrated in the same amount as native Exendin-4, in comparison with native Exendin-4 having very short half-life and thus poor in vivo stability.

1. A method of stabilizing a low-molecular-weight bioactive substance, comprising the steps of:
reacting a functional group on blood protein, which is selected from the group consisting of hydroxyl group (—OH), thiol group (—SH), amino group (—NH₂) and carboxyl group (—CO₂H), with a reactive group which is capable of forming a stable covalent bond with the functional group, to activate the blood protein; and
ex vivo reacting the activated blood protein with a low-molecular-weight bioactive substance having molecular weights of 100,000 or less and selected from the group consisting of a natural peptides, synthetic peptides, natural hormones, synthetic hormones, and raw materials for drugs, to form a stable covalent bond therebetween, wherein the reactive group is released after the covalent bond formation.

2. The method according to claim 1, wherein the low-molecular-weight bioactive substance is selected from the group consisting of insulinotropic peptides, glucagon family peptide hormones, and luteinizing hormone-releasing hormone (LHRH), and the blood protein is selected from the group consisting of albumin, transferrin, ferritin, and immunoglobulin.

3. The method according to claim 2, wherein the low-molecular-weight bioactive substance is selected from the group consisting of glucagons like peptide-1(GLP-1), exendin-3, exendin-4, and LHRH, and the blood protein is albumin.

4. The method according to claim 1, wherein the functional group on the blood protein is a thiol group (—SH), and the reactive group is a disulfanyl group capable of forming a stable disulfide bond with the thiol group.

5. The method according to claim 4, wherein the reactive group is selected from the group consisting of 2-pyridyl disulfanyl group, N-alkylpyridinium disulfanyl group, 5-nitro-2-pyridyl disulfanyl group, 3-nitro-thiophenyl disulfanyl, 1-piperido disulfanyl group, 3-cyano-propyl disulfanyl group, 2-thiouredyl disulfanyl group, 4-carboxylbenzyl disulfanyl group, 1-phenyl-1H-tetrazolyl disulfanyl group, 1-amino-2-naphthyl disulfanyl group, 3-carboxyl-6-pyridyl disulfanyl group, 2-benzothiazolyl disulfanyl group, and 4-nitro-thiophenyl disulfanyl group.

6. The method according to claim 5, wherein a functional group selected from the group consisting of a hydroxyl group (—OH), a thiol group (—SH), an amino group (—NH₂), and a carboxyl group (—CO₂H) is linked on the low-molecular-weight bioactive substance, and forms a stable covalent bond with the functional group on the blood protein activated by the reactive group.

7. The method according to claim 6, wherein both of the functional groups on the low-molecular-weight bioactive substance and on the blood protein are thiol groups, and the covalent bond is a stable disulfide covalent bond.

8. The method according to claim 6, wherein the functional group on the low-molecular-weight bioactive substance is linked to the low-molecular-weight bioactive substance through a linker group.

9. The method according to claim 8, wherein the linker group is selected from the group consisting of C1-C6 alkyl group, alkoxy group, cycloalkyl group, polycyclic group, aryl group, polyaryl group, substituted aryl group, heterocyclic group, substituted heterocyclic group and AE(E)ₙA ([2-(2-amino)-ethoxy](ethoxy)ₙacetic acid) (n is an integer between 0 and 2).

10. A bioactive substance-blood protein conjugate, wherein
the bioactive substance is a low-molecular-weight bioactive substance having the molecular weight of 100,000 or less and selected from the group consisting of a natural peptides, synthetic peptides, natural hormones, synthetic hormones, and raw materials for drugs;
the blood protein is activated by a reactive group capable of forming a sable covalent bond with the functional group on the blood protein;
the functional group on the blood protein is selected from the group consisting of a hydroxyl group (—OH), a thiol group (—SH), an amino group (—NH₂), and a carboxyl group (—CO₂H), and
a stable covalent bond is formed ex vivo between the bioactive substance and the functional group on the blood protein, whereby the stability of the bioactive substance is improved.

11. The bioactive substance-blood protein conjugate according to claim 10, wherein the low-molecular-weight bioactive substance is selected from the group consisting of insulinotropic peptides, glucagon family peptide hormones, and luteinizing hormone-releasing hormone (LHRH), and the blood protein is selected from the group consisting of albumin, transferrin, ferritin, and immunoglobulin.

12. The bioactive substance-blood protein conjugate according to claim 11, wherein the low-molecular-weight bioactive substance is selected from the group consisting of glucagons like peptide-1(GLP-1), exendin-3, exendin-4, and LHRH, and the blood protein is albumin.

13. The bioactive substance-blood protein conjugate according to claim 10, wherein the functional group on the blood protein is a thiol group, the reactive group is a disulfanyl group capable of forming a stable covalent bond with the functional group, and a stable disulfide covalent bond is formed between the functional group on the blood protein and the bioactive substance.

14. The bioactive substance-blood protein conjugate according to claim 13, wherein the reactive group is selected from the group consisting of 2-pyridyl disulfanyl group, N-alkylpyridinium disulfanyl group, 5-nitro-2-pyridyl disulfanyl group, 3-nitro-thiophenyl disulfanyl, 1-piperido disulfanyl group, 3-cyano-propyl disulfanyl group, 2-thiouredyl disulfanyl group, 4-carboxylbenzyl disulfanyl group, 1-phenyl-1H-tetrazolyl disulfanyl group, 1-amino-2-naphthyl disulfanyl group, 3-carboxyl-6-pyridyl disulfanyl group, 2-benzothiazolyl disulfanyl group, and 4-nitro-thiophenyl disulfanyl group.

15. The bioactive substance-blood protein conjugate according to claim 10, wherein a functional group selected from the group consisting of hydroxyl group (—OH), thiol group (—SH), amino group (—NH₂), and carboxyl group(—CO₂H) is linked on the low-molecular-weight bioactive substance, and forms a stable covalent bond with the functional group on the blood protein activated by the reactive group.

16. The bioactive substance-blood protein conjugate according to claim 15, wherein both of the functional groups on the low-molecular-weight bioactive substance and on the blood protein are thiol groups, and the covalent bond is a stable disulfide covalent bond.

17. The bioactive substance-blood protein conjugate according to claim 16, wherein the functional group on the low-molecular-weight bioactive substance is linked to the low-molecular-weight bioactive substance through a linker group.

18. The bioactive substance-blood protein conjugate according to claim 17, wherein the linker group is selected from the group consisting of C1-C6 alkyl group, alkoxy group, cycloalkyl group, polycyclic group, aryl group, polyaryl group, substituted aryl group, heterocyclic group, substituted heterocyclic group and AE(E)ₙA ([2-(2-amino)-ethoxy](ethoxy)ₙ acetic acid) (n is an integer between 0 and 2).

19. The bioactive substance-blood protein conjugate according to claim 18, wherein both of the functional groups on the low-molecular-weight bioactive substance and on the blood protein are thiol groups, the covalent bond is a stable disulfide covalent bond, and the linker is AEEEA.

20. A method of in vivo delivery of a bioactive substance, by administering the bioactive substance-blood protein conjugate according to any one of claims 10,1 wherein in vivo half-life and stability of the bioactive substance are improved.

21. (canceled)

22. A method of treating or preventing a disease on which a bioactive substance has a therapeutic effect, by administering the effective amount of the bioactive substance-blood protein conjugate according to any one of claims 10 to a patient in need of the administration thereof.

23. The method according to claim 22, wherein the disease is diabetes, prostate cancer, endometriosis, or uterus myoma.

24. (canceled)

25. (canceled)

26. A modified albumin, wherein a reactive group selected from the group consisting of 2-pyridyl disulfanyl group, N-alkylpyridinium disulfanyl group, 5-nitro-2-pyridyl disulfanyl group, 3-nitro-thiophenyl disulfanyl, 1-piperido disulfanyl group, 3-cyano-propyl disulfanyl group, 2-thiouredyl disulfanyl group, 4-carboxylbenzyl disulfanyl group, 1-phenyl-1H-tetrazolyl disulfanyl group, 1-amino-2-naphthyl disulfanyl group, 3-carboxyl-6-pyridyl disulfanyl group, 2-benzothiazolyl disulfanyl group, and 4-nitro-thiophenyl disulfanyl group is linked to cystein which is the 34^{th} amino acid of albumin, to activate the Cys³⁴ free thiol group of albumin.


SEQUENCE LISTING (20 sequences)

SEQ ID NO: 1 - protein, 31 aa, Artificial Sequence
  Synthetic
HAEGTFTSDVSSYLEGQAAKEFIAWLVKGRK

SEQ ID NO: 2 - protein, 31 aa, Artificial Sequence; identical to SEQ ID NO: 1
  Synthetic

SEQ ID NO: 3 - protein, 30 aa, Artificial Sequence
  Synthetic
HAEGTFTSDVSSYLEGQAAKEFIAWLVKGR

SEQ ID NO: 4 - protein, 30 aa, Artificial Sequence; identical to SEQ ID NO: 3
  Synthetic

SEQ ID NO: 5 - protein, 31 aa, Artificial Sequence; identical to SEQ ID NO: 1
  Synthetic

SEQ ID NO: 6 - protein, 31 aa, Artificial Sequence; identical to SEQ ID NO: 1
  Synthetic

SEQ ID NO: 7 - protein, 40 aa, Artificial Sequence
  Synthetic
HGEGTFTSDLSKQMEEEAVRLFIEWLKNGGPSSGAPPPSK

SEQ ID NO: 8 - protein, 40 aa, Artificial Sequence; identical to SEQ ID NO: 7
  Synthetic

SEQ ID NO: 9 - protein, 39 aa, Artificial Sequence
  Synthetic
HGEGTFTSDLSKQMEEEAVRLFIEWLKNGGPSSGAPPPS

SEQ ID NO: 10 - protein, 39 aa, Artificial Sequence; identical to SEQ ID NO: 9
  Synthetic

SEQ ID NO: 11 - protein, 40 aa, Artificial Sequence
  Synthetic
HSDGTFTSDLSKQMEEEAVRLFIEWLKNGGPSSGAPPPSK

SEQ ID NO: 12 - protein, 40 aa, Artificial Sequence; identical to SEQ ID NO: 11
  Synthetic

SEQ ID NO: 13 - protein, 39 aa, Artificial Sequence
  Synthetic
HSDGTFTSDLSKQMEEEAVRLFIEWLKNGGPSSGAPPPS

SEQ ID NO: 14 - protein, 39 aa, Artificial Sequence; identical to SEQ ID NO: 13
  Synthetic

SEQ ID NO: 15 - protein, 13 aa, Artificial Sequence
  Synthetic
EHWSYLLRPGSGK

SEQ ID NO: 16 - protein, 13 aa, Artificial Sequence
  Synthetic
EHWSYLLRPGSGK

SEQ ID NO: 17 - protein, 12 aa, Artificial Sequence
  Synthetic
EHWSYLLRPGGK

SEQ ID NO: 18 - protein, 12 aa, Artificial Sequence
  Synthetic
EHWSYLLRPGGK

SEQ ID NO: 19 - protein, 10 aa, Artificial Sequence
  Synthetic
EHWSYLLRPK

SEQ ID NO: 20 - protein, 10 aa, Artificial Sequence
  Synthetic
EHWSYLLRPK